(19) United States
(12) Patent Application Publication
(10) Pub. No.: US 2012/0190729 A1
(43) Pub. Date: Jul. 26, 2012

(54) MIRNA INHIBITION OF SIX1 EXPRESSION

(75) Inventors: Heide Ford, Aurora, CO (US); Anna Smith, Aurora, CO (US); David Poticha, Aurora, CO (US)

(73) Assignee: The Regents of The University of Colorado A Body Corporate, Denver, CO (US)

(21) Appl. No.: 13/387,259

(22) PCT Filed: Jul. 27, 2010
(86) PCT No.: PCT/US10/43354

§ 371 Date: Apr. 9, 2012

Related U.S. Application Data
(60) Provisional application No. 61/228,726, filed on Jul. 27, 2009.

(51) Int. Cl. A61K 31/7088; C40B 30/00; C12Q 1/68; A61P 35/00; A61P 35/02

(52) U.S. Cl. 514/44 A; 435/6.12; 506/7

(57) ABSTRACT

The invention provides a method of treating cancer in a subject comprising administering to said subject an miRNA that inhibits Six1. In some embodiments, the invention further provides for the administration of a second cancer therapy to the subject.

This application claims benefit of priority to U.S. Provisional Application Ser. No. 61/228,726, filed on Jul. 27, 2009, the entire content of which is hereby incorporated by reference.

This invention was made with government support under Grant No. CA095277 awarded by the National Institutes of Health. The government has certain rights in the invention.

### BACKGROUND OF THE INVENTION

I. Field of the Invention

The present invention relates generally to the fields of oncology, molecular biology, and medicine. More particularly, the invention relates to the use of miRNAs to inhibit expression of Six1, including in the context of cancer treatments.

II. Description of Related Art

Cancer shares many common properties with normal development. During normal development of an organ, genes are activated to stimulate the proliferation and survival of progenitor cells, as well as to stimulate migration, invasion, and neovascularization. These genes are usually down-regulated once organ development is completed. In cancer, the same genes are often re-activated, stimulating inappropriate proliferation, survival, migration, invasion, and neovascularization. Thus, there is a need for treatments that inhibit the expression of such genes after organ development is completed.

Homeobox gene superfamily transcription factors act as master regulators of development through their ability to activate or repress a diverse range of downstream target genes. Numerous families exist within the homeobox gene superfamily, and are classified on the basis of conservation of their homeodomains as well as additional motifs that contribute to DNA binding and to interactions with other proteins. Members of one such family, the Six family, form a transcriptional complex with Eya and Dach proteins, and together these proteins make up part of the retinal determination network first identified in Drosophila. This network is highly conserved in both invertebrate and vertebrate species, where it influences the development of numerous organs in addition to the eye, primarily through regulation of cell proliferation, survival, migration, and invasion.

Mutations in the Six family member Six1 have been identified in a variety of human genetic disorders, demonstrating their critical role in human development. In addition, aberrant expression of Six1 occurs in numerous human tumors, and in particular, play a causal role both in tumor initiation and in metastasis. Emerging evidence for the importance of Six family members and their cofactors in numerous human tumors suggests that targeting of this complex may be a novel and powerful means to inhibit both tumor growth and progression. Further research is needed to identify appropriate therapeutics that target Six1.

### SUMMARY OF THE INVENTION

In one aspect, the invention provides a method of treating cancer in a subject comprising administering to said subject an effective amount of at least a first Six1 miRNA. The miRNA may target the Six1 3′-UTR, such as miR-185, miR-571 or miR-639. The method may further comprise administering at least two or all three of miR-185, miR-571 and miR-639. The miRNA may be administered topically, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctivally, intravesicularly, mucosally, intrapericardially, intraumbilically, orally, by inhalation, by injection, by infusion, by continuous infusion, by localized perfusion bathing target cells directly, via a catheter, or via a lavage. The method miRNA may comprise one or more stabilized non-natural nucleotides. The method may further comprise administering to said subject an effective amount of an antagomir selected from miR-106b, miR-93, or miR-25, or two or all three of these antagomirs.

The miRNA may be administered more than once. The miRNA may be administered by provision to said subject of an expression construct, such as a viral expression construct or a non-viral expression construct. The miRNA may be admininstered in a lipid vehicle. The method may further comprise administering to said patient a second cancer therapy, such as radiotherapy, immunotherapy, chemotherapy, hormonal therapy or gene therapy. The cancer may be breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia. The method may further comprise administering to said subject an antagomir of an miRNA that upregulates Six1 expression, and the antagomir may be administered more than once.

In another embodiment, there is provided a method of identifying a Six1-involved cancer in a subject comprising (a) obtaining a sample from said subject; (b) assaying for the presence of one or more miRNAs in said sample selected from the group consisting of miR-106b, miR-93, miR-25, miR-375 and miR-622; wherein an increase in said sample of one or more of miR-106b, miR-93, or miR-25, or a decrease in one or both of miR-375 or miR-622, as compared to a non-cancerous sample, indicates that said subject has a Six1-involved cancer. Each of miR-106b, miR-93 and miR-25 may be assayed, each of miR-375 and miR-622 may be assayed, or each of miR-106b, miR-93, miR-25, miR-375 and miR-622 may be assayed. The assaying may comprise microarray hybridization. Each of miR-106b, miR-93 and miR-25 may be increased, or each of miR-375 and miR-622 may be decreased. The method may further comprise administering to said subject an effective amount of at least a first Six1 miRNA when at least one of miR-106b, miR-93 and miR-25 are increased. The method may also further comprise administering to said subject an effective amount of at least a first Six1 miRNA when at least one of miR-375, and miR-622 are increased. The sample may be biopsy sample or a tumor resection specimen. The methods may also apply to other miRNAs set forth in Table 1.

In yet another embodiment, there is provided a method of identifying a subject with cancer comprising (a) obtaining a sample from said subject; (b) assaying for the presence of one or more miRNAs in said sample selected from the group consisting of miR-106b, miR-93, miR-25, miR-375 and miR-622; wherein an increase in said sample of one or more of miR-106b, miR-93, or miR-25, or a decrease in one or both of miR-375 or miR-622, as compared to a non-cancerous sample, indicates that said subject has cancer. Each of miR-106b, miR-93 and miR-25 may be assayed, each of miR-375 and miR-622 may be assayed, or each of miR-106b, miR-93, miR-25, miR-375 and miR-622 may be assayed. The assaying may comprise microarray hybridization. Each of miR-106b, miR-93 and miR-25 may be increased, or each of miR-375 and miR-622 may be decreased. The method may further comprise administering to said subject an effective amount of at least a first Six1 miRNA when at least one of miR-106b, miR-93 and miR-25 are increased. The method may also further comprise administering to said subject an effective amount of at least a first Six1 miRNA when at least one of miR-375, and miR-622 are increased. The sample may be biopsy sample or a tumor resection specimen. The methods may also apply to other miRNAs set forth in Table 1.

In still yet another embodiment, there is provided a method of treating cancer in a subject comprising administering to said subject an effective amount of an antagomir of miR-106b, miR-93, or miR-25. The method may further comprise administering at least two of or all three of miR-106b, miR-93, and/or miR-25. The antagomir may be administered topically, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctivally, intravesicularly, mucosally, intrapericardially, intraumbilically, orally, by inhalation, by injection, by infusion, by continuous infusion, by localized perfusion bathing target cells directly, via a catheter, or via a lavage. The antagomir may be administered more than once. The antagomir may be administered by provision to said subject of an expression construct, such as a viral expression construct or a non-viral expression construct. The antagomir may be admininstered in a lipid vehicle. The method may further comprise administering to said patient a second cancer therapy. The cancer may be breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia. The method may further comprise administering miR-185, miR-571 or miR-639 to said subject, or administering at least two of miR-185, miR-571 and miR-639, or administering all three of miR-185, miR-571 and miR-639.

Any embodiment discussed with respect to one aspect of the invention applies to other aspects of the invention as well.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIG. 1—miRNA predictions for regulation of Six1. miRNA prediction databases were used to predict miRNAs that may regulate Six1 based on seed match sites in the 3′UTR of Six1. These are the top hits from TargetScan database (one from Pictar), which were used for analysis.

FIGS. 2A-B. Selected miRNAs Target Six1 3′UTR (FIG. 2A) Six1 3′UTR was cloned into the psi-CHECK2 dual luciferase vector (Six1-Luc) and co-transfected into HEK293 cells with miRNA mimics for hsa-miR-185 and hsa-miR-639 (red bars). Six1 3′UTR-Check2 vectors were also mutated at the selected miRNA seed regions (Mut-Luc) and co-transfected with miRNA mimics for comparison (green bars). Luciferase reading was taken 48 hours post-transfection, where Renilla is normalized against Firefly Luciferase. (FIG. 2B) The Six1-Luc vector was co-transfected with miRNA mimics for hsa-miR-22, hsa-miR-758, and hsa-miR-571, and luciferase readings were taken. No mutated seed matches have been compared for this group yet.

FIGS. 3A-D. Expression of Six1 and selected miRNAs across breast cancer cell lines using qPCR. RNA was extracted from selected cell lines and endogenous expression of Six1 (FIG. 3A), miR-639 (FIG. 3B), miR-185 (FIG. 3C), and miR-571 (FIG. 3D) were compared across 3 normal breast epithelial cells (yellow bars) and 8 breast cancer cells (blue bars) using qRT-PCR. Six 1 expression is normalized to Cyclophilin B, while miRNA expression is normalized to U6 RNA. Results show the relative quantification using ddCT method.

FIGS. 4A-C. miR-185, miR-639, and miR-571 repress Six1 protein and mRNA expression in BT549 cells. miRNA mimics for miR-185, 639, and 571 were transfected into BT549 cells, which express high endogenous levels of Six1. (FIG. 4A) Expression of mature miRNAs after mimic transfection were assayed by qPCR. Ct values were normalized to U6 RNA levels and calculated with ddCT to show relative expression. (FIG. 4B) Western Blot showing protein levels of Six1 after mimic transfections in BT549 cells. Neg mimic is a nontargeting miRNA, and NMuMG cell line was used as a positive control for Six1 protein. (FIG. 4C) qPCR showing correlative Six1 mRNA expression after mimic transfections in BT549 cells. Ct values were normalized to CyclophilinB and calculated with ddCT showing relative expression.

FIGS. 5A-B. Validation of microarray miRNAs with qRT-PCR. (FIG. 5A) An additional miRNA was included in the validation screen, hsa-miR-93, as it was found that this miRNA sits in an intronic cluster of the MCM7 gene with miR-106b and miR-25 (the 106b-25 cluster). (FIG. 5B) From the top miRNA candidates on the microarray, 4 miRNAs recapitulated the -fold difference on a qRT-PCR assay between MCF7-CTRL and MCF7-Six1 cells. Bars represent the average of 3 biological replicates with standard error of the mean. Additionally, miR-93 also followed the expression of the other miRNAs in the 106b-25 cluster, suggesting that Six1 may regulate this cluster of miRNAs.

FIGS. 6A-B. miR-106b-25 cluster correlates with endogenous Six1 expression. (FIG. 6A) qRT-PCR with the isogenic 21T series of breast cancer cells shows high Six1 in the 4 carcinogenic cells (21PT, 21NT, 21MT1, 21MT2) as compared to the normal breast cell (16N). The miR-106b-25 cluster miRNAs, as also shown by qRT-PCR, all correlate with the endogenous Six1 expression in the 21T samples. (FIG. 6B) Six1 was knocked down in 21PT cells at two different concentrations (50 nm, 100 nm) (Left). The 106b-25 miRNAs also decrease in expression with Six1 knockdown as shown by qRT-PCR. Six1 assays were normalized to CyclophilinB, while miRNA assays were normalized to U6 RNA.

FIGS. 7A-D: The miR-106b-25 cluster may activate TGFbeta signaling. (FIG. 7A) Introduction of a stable vector expressing the miR-106b-25 cluster (miRex-Cluster) vs. a non-targeting control (miRex-CTRL) increases expression of TβR-I protein by western blot of whole cell lysates, and (FIG. 7B) increases expression of p-Smad3 protein in nuclear extracts. (FIG. 7C) Transient expression of the cluster in MCF7 cells also have increased TGFβ transcriptional activity by 3TP luciferase assay, and (FIG. 7D) increased β-catenin transcriptional activity by TOP-flash luciferase assay. Luciferase assays are normalized to Renilla luciferase activity.

FIGS. 8A-C: The miR106b-25 cluster is sufficient to induce cancer stem cell phenotype as seen with Six1 overexpression. (FIG. 8A) The number of mammospheres formed in Mammosphere formation assays per 1000 cells plated for each. MCF7 cells overexpressing both Six1 and Cluster have similar increase in mammosphere formation (FIG. 8B) Flow Cytometry for CD24 low CD44 hi population in the same Cluster overexpressing cells as in A. (FIG. 8C) Serial dilution of cluster overexpressing cells vs. control cells injected into mammary fat pad of mice. Number represent observed tumors out of 10 mice per group.

### DESCRIPTION OF ILLUSTRATIVE EMBODIMENTS

Cancer shares many common properties with normal development. During normal development of an organ, genes are activated to stimulate the proliferation and survival of progenitor cells, as well as to stimulate migration, invasion, and neovascularization. These genes are usually down-regulated once organ development is completed. In cancer, the same genes are often re-activated, stimulating inappropriate proliferation, survival, migration, invasion, and neovascularization. The homeobox gene and transcription factor Six1 plays a critical role in the development of numerous organs through its ability to increase proliferation and decrease apoptosis, leading to an expansion of progenitor cell populations (Kawakami et al., 2000; Xu et al., 2003; Zheng et al., 2003; Laclef et al., 2003a; Laclef et al., 2003b; Ozaki et al., 2004). Six1 expression is undetectable or low in normal adult breast tissue, but it is over-expressed in 50% primary breast tumors and 90% metastatic lesions (Coletta et al., 2004; Reichenberger et al., 2005). Six1 over-expression is linked to enhanced cellular proliferation, transformation, increased tumor volume, epithelial to mesenchymal transition, and metastasis in breast cancer (Coletta et al., 2004; Coletta et al., 2008). Further, epithelial-to-mesenchymal transition (EMT) is heavily associated with metastasis. The relocalization of Ecad and β-catenin is associated with EMT.

Data shows that overexpression of Six1 can transform immortalized, but otherwise normal, mammary epithelial cells, leading to highly aggressive tumors in vivo. Furthermore, data in which Six1 was overexpressed specifically in the mammary gland show that Six1 induces hyperplasia and tumor formation in vivo. Together, these data indicated that Six1 is involved in tumor initiation. The higher percentage of Six1 over-expression in metastatic breast lesions indicates that it may also play an important role in breast tumor metastasis. Examination of more than 130 breast cancers demonstrates that Six1 overexpression correlates significantly with positive lymph node status (p<0.05). Six 1 overexpression in tumorigenic, but non-metastatic MCF7 cells leads to both lymphatic and bone metastasis in a mouse orthotopic breast cancer model. Six1 expression leads to expansion of CD24+/CD29^{hi} mammary stem/progenitor cells in transgenic mice, and to the expansion of CD44+/CD24lo cancer stem cells in MCF7 mammary carcinoma cells. These studies indicate that Six1 is a powerful oncogene that can not only induce tumorigenesis, but can also cause metastasis.

Furthermore, Six1 is overexpressed in both ovarian carcinoma and hepatacellular carcinoma, and its expression correlates with worsened survival in both cancer types (Ng et al., 2006). Six1 is also overexpressed in rhabdomyosarcomas where its expression correlates with advanced tumor stage and where it is shown to be critical for metastasis (Li et al., 2002; Khan et al., 1999; Yu et al., 2004). Six1 is amplified in a small percentage (about 5%) of human breast cancers and is overexpressed in Wilms' Tumor (Li et al., 2002). These data suggest that Six1 plays a role in the progression of many tumor types. Because Six1 is overexpressed in multiple cancers, and because it is an embryonic gene whose expression is absent in most differentiated adult tissues, it is an ideal drug target whose inactivation will inhibit tumor cell proliferation, survival, and metastasis with limited side effects. Importantly, RNA interference against Six1 decreases cancer cell proliferation and metastases in several different models of cancer. Similarly, as Six1 influences multiple stages of the tumorigenic process, targeting the Six1 transcriptional complex has the therapeutic potential to inhibit breast cancer both at early and later stages of disease progression.

The inventors have now identified several miRNAs that target the 3′ UTR of the Six1 gene and down-regulate Six1 expression. They also have identified miRNAs that correlate with alterations in Six1 expression, and thus can be used to identify Six1-involved cancers, as well as prove diagnostic of cancer in their own right.

### I. Six1

Six1 belongs to the Six family of homeobox genes (Six1-6) encoding transcription factors that play vital roles in the development of many organs (Kawakami et al., 2000). Six1-6 share a DNA binding homeodomain (HD) and a Six domain (SD) responsible for co-activator binding (Kawakami et al., 2000). In particular, Six1 plays a role in cell growth, cell survival and cell migration during normal cell development. Six1 plays a critical role in the onset and progression of a significant proportion of breast and other cancers, but has never before been clinically targeted. The Six1 homeobox gene encodes a transcription factor that is crucial for the development of many organs but is down-regulated after organ development is complete. Its expression is low or undetectable in normal adult breast tissue but the gene is over-expressed in 50% of primary breast tumors and 90% of metastatic lesions. Examination of public microarray databases containing more than 535 breast cancer samples demonstrates that Six1 levels correlate significantly with shortened time to relapse, shortened time to metastasis, and decreased overall survival. In addition, Six1 overexpression correlates with adverse outcomes in numerous other cancers, including ovarian, hepatocellular carcinoma, and rhabdomyosarcoma. Using mouse models of mammary cancer, it was recently demonstrated that over-expression of Six1 results in enhanced proliferation, transformation, increased tumor volume, and metastasis. Importantly, RNA interference against Six1 decreases cancer cell proliferation and metastases in several different cancer models.

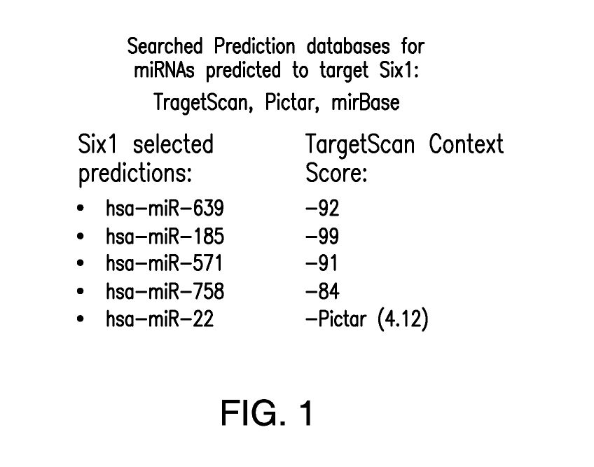

Six1 was shown to bind tightly to the MEF3 motif (TCAGGTT) (Spitz et al., 1998). This sequence is different from the TAAT core sequence bound by the canonical HD, likely due to the fact that the HD in Six1 differs from the “classic” HD at two highly conserved residues contacting DNA. The Six type HD is believed to confer a unique DNA binding specificity to the Six family members that differs from the TAAT core in the classic HD. However, the consensus Six1 recognition sequence remains unknown. A limited number of potential Six1 targets are identified (Kawakami et al., 1996; Spitz et al., 1998; Ando et al., 2005) and, indeed, none of them contain the TAAT core. Interestingly, these targets do not share an obvious consensus sequence, possibly due to the limited number of sequences analyzed. Recently, an ideal Six1 DNA binding sequence (TGATAC) was identified using combined bioinformatic and biologic approaches (Noyes et al., 2008; Berger et al., 2008). The Six1 target most relevant to breast tumorigenesis is the cyclin A1 promoter (Coletta et al., 2004). The transcriptional up-regulation of cyclin A1 by Six1 leads to an increase in proliferation in mammary carcinoma cells and Six1 mediated cell cycle progression is dependent on cyclin A1 (Coletta et al., 2004). In addition to the HD, the Six family members contain a conserved and novel Six-domain (SD) (FIG. 1) (Oliver et al., 1995). The SD contributes to DNA binding as well as to protein interaction with cofactors (Kawakami et al., 2000; Oliver et al., 1995).

Six1 does not have an intrinsic activation or repression domain and requires the Eya coactivator proteins to activate transcription. The Eya proteins utilize their intrinsic phosphatase activity to switch the Six1 transcriptional complex from a repressor to an activator complex. The Six1-Eya interaction is essential for proliferation during embryonic development, and both Six1 and Eya2 have been independently implicated in the same types of cancer. Because the Eya co-activator contains a unique protein phosphatase domain whose activity is required to activate Six1, it may serve as a novel anti-cancer drug target. Eya knockout mice phenocopy Six1 knockout mice (Xu et al., 1999). Six1's activity on cellular proliferation was also found to be dependent on Eya (Li et al., 2003). As Six1 contributes to breast tumorigenesis by stimulating cellular proliferation, the interaction between Eya and Six1 may be critical for Six1-mediated tumorigenesis.

### II. miRNAs

A. Background

In 2001, several groups used a novel cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are nonetheless distinct.

miRNAs thus far observed have been approximately 21-22 nucleotides in length and they arise from longer precursors, which are transcribed from non-protein-encoding genes. See review of Carrington et al. (2003). The precursors form structures that fold back on each other in self-complementary regions; they are then processed by the nuclease Dicer in animals or DCL1 in plants. miRNA molecules interrupt translation through precise or imprecise base-pairing with their targets. miRNAs are transcribed by RNA polymerase II and can be derived from individual miRNA genes, from introns of protein coding genes, or from poly-cistronic transcripts that often encode multiple, closely related miRNAs. Pre-miRNAs, generally several thousand bases long are processed in the nucleus by the RNase Drosha into 70- to 100-nt hairpin-shaped precursors. Following transport to the cytoplasm, the hairpin is further processed by Dicer to produce a double-stranded miRNA. The mature miRNA strand is then incorporated into the RNA-induced silencing complex (RISC), where it associates with its target mRNAs by base-pair complementarity. In the relatively rare cases in which a miRNA base pairs perfectly with an mRNA target, it promotes mRNA degradation. More commonly, miRNAs form imperfect heteroduplexes with target mRNAs, affecting either mRNA stability or inhibiting mRNA translation.

The 5′ portion of a miRNA spanning bases 2-8, termed the ‘seed’ region, is especially important for target recognition (Krenz and Robbins, 2004; Kiriazis and Krania, 2000). The sequence of the seed, together with phylogenetic conservation of the target sequence, forms the basis for many current target prediction models. Although increasingly sophisticated computational approaches to predict miRNAs and their targets are becoming available, target prediction remains a major challenge and requires experimental validation. Ascribing the functions of miRNAs to the regulation of specific mRNA targets is further complicated by the ability of individual miRNAs to base pair with hundreds of potential high and low affinity mRNA targets and by the targeting of multiple miRNAs to individual mRNAs.

The first miRNAs were identified as regulators of developmental timing in C. elegans, suggesting that miRNAs, in general, might play decisive regulatory roles in transitions between different developmental states by switching off specific targets (Fatkin et al., 2000; Lowes et al., 1997). However, subsequent studies suggest that miRNAs, rather than functioning as on-off “switches,” more commonly function to modulate or fine-tune cell phenotypes by repressing expression of proteins that are inappropriate for a particular cell type, or by adjusting protein dosage. miRNAs have also been proposed to provide robustness to cellular phenotypes by eliminating extreme fluctuations in gene expression (Miyata et al., 2000).

Research on microRNAs is increasing as scientists are beginning to appreciate the broad role that these molecules play in the regulation of eukaryotic gene expression. The two best understood miRNAs, lin-4 and let-7, regulate developmental timing in C. elegans by regulating the translation of a family of key mRNAs (reviewed in Pasquinelli, 2002). Several hundred miRNAs have been identified in C. elegans, Drosophila, mouse, and humans. As would be expected for molecules that regulate gene expression, miRNA levels have been shown to vary between tissues and developmental states. In addition, one study shows a strong correlation between reduced expression of two miRNAs and chronic lymphocytic leukemia, providing a possible link between miRNAs and cancer (Calin et al., 2002). Although the field is still young, there is speculation that miRNAs could be as important as transcription factors in regulating gene expression in higher eukaryotes.

There are a few examples of miRNAs that play critical roles in cell differentiation, early development, and cellular processes like apoptosis and fat metabolism. lin-4 and let-7 both regulate passage from one larval state to another during C. elegans development (Ambros, 2003). mir-14 and bantam are drosophila miRNAs that regulate cell death, apparently by regulating the expression of genes involved in apoptosis (Brennecke et al., 2003, Xu et al., 2003). MiR14 has also been implicated in fat metabolism (Xu et al., 2003). Lsy-6 and miR-273 are C. elegans miRNAs that regulate asymmetry in chemosensory neurons (Chang et al., 2004). Another animal miRNA that regulates cell differentiation is miR-181, which guides hematopoietic cell differentiation (Chen et al., 2004). These molecules represent the full range of animal miRNAs with known functions. Enhanced understanding of the functions of miRNAs will undoubtedly reveal regulatory networks that contribute to normal development, differentiation, inter- and intra-cellular communication, cell cycle, angiogenesis, apoptosis, and many other cellular processes. Given their important roles in many biological functions, it is likely that miRNAs will offer important points for therapeutic intervention or diagnostic analysis.

Characterizing the functions of biomolecules like miRNAs often involves introducing the molecules into cells or removing the molecules from cells and measuring the result. If introducing a miRNA into cells results in apoptosis, then the miRNA undoubtedly participates in an apoptotic pathway. Methods for introducing and removing miRNAs from cells have been described. Two recent publications describe antisense molecules that can be used to inhibit the activity of specific miRNAs (Meister et al., 2004; Hutvagner et al., 2004). Another publication describes the use of plasmids that are transcribed by endogenous RNA polymerases and yield specific miRNAs when transfected into cells (Zeng et al., 2002). These two reagent sets have been used to evaluate single miRNAs.

B. miR-185

The sequence for miR-185 is provided as SEQ ID NO:1. It was one of a set of 4 miRNAs (miR-28, miR-185, miR-27, and let-7f-2) found to be significantly up-regulated in renal cell carcinoma (p<0.05) compared to normal kidney. It was also one of 10 miRNAs (miR-223, miR-26b, miR-221, miR-103-1, miR-185, miR-23b, miR-203, miR-17-5p, miR-23a, miR-205) that were significantly up-regulated in bladder cancers (p<0.05) compared to normal bladder mucosa (Gottardo et al., 2007).

C. miR-639

The sequence for miR-639 is provided as SEQ ID NO:2.

D. miR-571

The sequence for miR-571 is provided as SEQ ID NO:3.

E. Surrogates

The inventors have also identified a number of miRNAs that appear to be regulated by Six1, or at least connected to the expression of Six1 such that their levels fluctuate along with levels of Six1. miR-106b (SEQ ID NO:4), miR-93 (SEQ ID NO:5), and miR-25 (SEQ ID NO:6) are all observed to be increased where Six1 is increased, and both miR-375 (SEQ ID NO:7) and miR-622 (SEQ ID NO:8) are decreased with an increase in Six1 expression.

F. Antagomirs

In certain embodiments, it may be desirable to also employ inhibitors of endogenous miRNAs that modulate Six1 expression, particularly those that increase Six 1 expression. It is specifically contemplated that miRNAs exist that counterbalance the effects of the inhibitory miRNAs disclosed herein, and inhibition of these miRNAs may help prevent a compensatory increase in the expression when Six1 is inhibited by miR-189, miR-639 and miR-571. In general, such inhibitors of miRNAs take the form of “antagomirs,” short, chemically-engineered single-stranded oligonucleotides complementary to miRNAs that block their function (Krützfeldt et al., 2005). Other approaches include inhibition of miRNAs with antisense 2′-β-methyl (2′-OMe) oligoribonucleotides and small interfering double-stranded RNAs (siRNAs) engineered with certain “drug-like” properties (chemical modifications for stability; cholesterol conjugation for delivery) (Krützfeldt et al., 2005).

G. Synthesis and Alternative Nucleic Acid Chemistries

Oligonucleotides are chemically synthesized using nucleoside phosphoramidites. A phosphoramidite is a derivative of natural or synthetic nucleoside with protection groups added to its reactive exocyclic amine and hydroxy groups. The naturally occurring nucleotides (nucleoside-3′-phosphates) are insufficiently reactive to afford the synthetic preparation of oligonucleotides. A dramatically more reactive (2-cyanoethyl) N,N-diisopropyl phosphoramidite group is therefore attached to the 3′-hydroxy group of a nucleoside to form nucleoside phosphoramidite. The protection groups prevent unwanted side reactions or facilitate the formation of the desired product during synthesis. The 5′-hydroxyl group is protected by DMT (dimethoxytrityl) group, the phosphite group by a diisopropylamino (iPr2N) group and a 2-cyanoethyl (OCH₂CH₂CN) group. The nucleic bases also have protecting groups on the exocyclic amine groups (benzoyl, acetyl, isobutyryl, or many other groups). In RNA synthesis, the 2′ group is protected with a TBDMS (t-butyldimethylsilyl) group or with a TOM (t-butyldimethylsilyloxymethyl) group. With the completion of the synthesis process, all the protection groups are removed.

Whereas enzymes synthesize DNA in a 5′ to 3′ direction, chemical DNA synthesis is done backwards in a 3′ to 5′ reaction. Based on the desired nucleotide sequence of the product, the phosphoramidites of nucleosides A, C, G, and T are added sequentially to react with the growing chain in a repeating cycle until the sequence is complete. In each cycle, the product's 5′-hydroxy group is deprotected and a new base is added for extension. In solid-phase synthesis, the oligonucleotide being assembled is bound, via its 3′-terminal hydroxy group, to a solid support material on which all reactions take place. The 3′ group of the first base is immobilized via a linker onto a solid support (most often, controlled pore glass particles or macroporouspolystyrene beads). This allows for easy addition and removal of reactants. In each cycle, several solutions containing reagents required for the elongation of the oligonucleotide chain by one nucleotide residue are sequentually pumped through the column from an attached reagent delivery system and removed by washing with an inert solvent.

Antagomirs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism. Modifications can also increase sequence specificity, and consequently decrease off-site targeting. In one embodiment, the antagomir includes a non-nucleotide moiety, e.g., a cholesterol moiety. The non-nucleotide moiety can be attached to the 3′ or 5′ end of the oligonucleotide agent.

A wide variety of well-known, alternative oligonucleotide chemistries may be used (see, e.g., U.S. Patent Publications 2007/0213292, 2008/0032945, 2007/0287831, etc.), particularly single-stranded complementary oligonucleotides comprising 2′ methoxyethyl, 2′-fluoro, and morpholino bases (see e.g., Summerton and Weller, 1997). The oligonucleotide may include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). Also contemplated are locked nucleic acid (LNA) and peptide nucleic acids (PNA).

Peptide nucleic acids (PNAs) are nonionic DNA mimics that have outstanding potential for recognizing duplex DNA (Kaihatsu et al., 2004; Nielsen et al., 1991). PNAs can be readily synthesized and bind to complementary sequences by standard Watson-Crick base-pairing (Egholm et al., 1993), allowing them to target any sequence within the genome without the need for complex synthetic protocols or design considerations. Strand invasion of duplex DNA by PNAs is not hindered by phosphate-phosphate repulsion and is both rapid and stable (Kaihatsu et al., 2004; Nielsen et al., 1991). Applications for strand invasion by PNAs include creation of artificial primosomes (Demidov et al., 2001), inhibition of transcription (Larsen and Nielsen, 1996), activation of transcription (Mollegaard et al., 1994), and directed mutagenesis (Faruqi et al., 1998). PNAs would provide a general and potent strategy for probing the structure and function of chromosomal DNA in living systems if their remarkable strand invasion abilities could be efficiently applied inside cells.

Strand invasion by PNAs in cell-free systems is most potent at sequences that are partially single-stranded (Bentin and Nielsen, 1996; Zhang et al., 2000). Assembly of RNA polymerase and transcription factors into the pre-initiation complex on DNA induces the formation of a structure known as the open complex that contains several bases of single-stranded DNA (Holstege et al., 1997; Kahl et al., 2000). The exceptional ability of PNAs to recognize duplex DNA allows them to intercept the open complex of an actively transcribed gene without a requirement for preincubation. The open complex is formed during transcription of all genes and PNAs can be synthesized to target any transcription initiation site. Therefore, antigene PNAs that target an open complex at a promoter region within chromosomal DNA would have the potential to be general tools for controlling transcription initiation inside cells.

A locked nucleic acid (LNA), often referred to as inaccessible RNA, is a modified RNA nucleotide (Elmen et al., 2008). The ribose moiety of an LNA nucleotide is modified with an extra bridge connecting the 2′ and 4′ carbons. The bridge “locks” the ribose in the 3′-endo structural conformation, which is often found in the A-form of DNA or RNA. LNA nucleotides can be mixed with DNA or RNA bases in the oligonucleotide whenever desired. Such oligomers are commercially available. The locked ribose conformation enhances base stacking and backbone pre-organization. This significantly increases the thermal stability (melting temperature) of oligonucleotides (Kaur et al., 2006). LNA bases may be included in a DNA backbone, by they can also be in a backbone of LNA, 2′-O-methyl RNA, 2′-methoxyethyl RNA, or 2′-fluoro RNA. These molecules may utilize either a phosphodiester or phosphorothioate backbone.

Other oligonucleotide modifications can be made to produce oligonucleotides. For example, stability against nuclease degradation has been achieved by introducing a phosphorothioate (P═S) backbone linkage at the 3′ end for exonuclease resistance and 2′ modifications (2′-OMe, 2′-F and related) for endonuclease resistance (WO 2005115481; Li et al., 2005; Choung et al., 2006). A motif having entirely of 2′-O-methyl and 2′-fluoro nucleotides has shown enhanced plasma stability and increased in vitro potency (Allerson et al., 2005). The incorporation of 2′-O-Me and 2′-O-MOE does not have a notable effect on activity (Prakash et al., 2005).

Sequences containing a 4′-thioribose modification have been shown to have a stability 600 times greater than that of natural RNA (Hoshika et al, 2004). Crystal structure studies reveal that 4′-thioriboses adopt conformations very similar to the C3′-endo pucker observed for unmodified sugars in the native duplex (Haeberli et al., 2005). Stretches of 4′-thio-RNA were well tolerated in both the guide and nonguide strands. However, optimization of both the number and the placement of 4′-thioribonucleosides is necessary for maximal potency.

In the boranophosphate linkage, a non-bridging phosphodiester oxygen is replaced by an isoelectronic borane (BH3-) moiety. Boranophosphate siRNAs have been synthesized by enzymatic routes using T7 RNA polymerase and a boranophosphate ribonucleoside triphosphate in the transcription reaction. Boranophosphate siRNAs are more active than native siRNAs if the center of the guide strand is not modified, and they may be at least ten times more nuclease resistant than unmodified siRNAs (Hall et al., 2004; Hall et al., 2006).

Certain terminal conjugates have been reported to improve or direct cellular uptake. For example, NAAs conjugated with cholesterol improve in vitro and in vivo cell permeation in liver cells (Rand et al., 2005). Soutschek et al. (2004) have reported on the use of chemically-stabilized and cholesterol-conjugated siRNAs have markedly improved pharmacological properties in vitro and in vivo. Chemically-stabilized siRNAs with partial phosphorothioate backbone and 2′-O-methyl sugar modifications on the sense and antisense strands (discussed above) showed significantly enhanced resistance towards degradation by exo- and endonucleases in serum and in tissue homogenates, and the conjugation of cholesterol to the 3′ end of the sense strand of an oligonucleotides by means of a pyrrolidine linker does not result in a significant loss of gene-silencing activity in cell culture. These study demonstrates that cholesterol conjugation significantly improves in vivo pharmacological properties of oligonucleotides.

U.S. Patent Publication 2008/0015162, incorporated herein by reference, provide additional examples of nucleic acid analogs useful in the present invention.

The following excerpts are derived from that document and are exemplary in nature only.

In certain embodiments, oligomeric compounds comprise one or more modified monomers, including 2′-modified sugars, such as BNA's and monomers (e.g., nucleosides and nucleotides) with 2′-substituents such as allyl, amino, azido, thio, O-allyl, O—C₁-C₁₀ alkyl, —OCF₃, O—(CH₂)₂—O—CH₃, 2′—O(CH₂)₂SCH₃, O—(CH₂)₂—0—N(Rₘ)(Rₙ), or O—CH₂—C(═O)—N(Rₘ)(Rₙ), where each Rₘ and Rₙ is, independently, H or substituted or unsubstituted C₁-C₁₀ alkyl.

In certain embodiments, the oligomeric compounds including, but no limited to short oligomers of the present invention, comprise one or more high affinity monomers provided that the oligomeric compound does not comprise a nucleotide comprising a 2′-O(CH₂)ₙH, wherein n is one to six. In certain embodiments, the oligomeric compounds including, but no limited to short oligomers of the present invention, comprise one or more high affinity monomer provided that the oligomeric compound does not comprise a nucleotide comprising a 2′-OCH₃ or a 2′-O(CH₂)₂OCH₃. In certain embodiments, the oligomeric compounds comprise one or more high affinity monomers provided that the oligomeric compound does not comprise a α-L-methyleneoxy (4′-CH₂—O-2′) BNA and/or a β-D-methyleneoxy (4′-CH₂—O-2′) BNA.

Certain BNAs have been prepared and disclosed in the patent literature as well as in scientific literature (Singh et al., 1998; Koshkin et al., 1998; Wahlestedt et al., 2000; Kumar et al., 1998; WO 94/14226; WO 2005/021570; Singh et al, 1998; examples of issued US patents and published applications that disclose BNAs include, for example, U.S. Pat. Nos. 7,053,207; 6,268,490; 6,770,748; 6,794,499; 7,034,133; and 6,525,191; and U.S. Patent Publication Nos. 2004/0171570; 2004/0219565; 2004/0014959; 2003/0207841; 2004/0143114; and 2003/0082807.

Also provided herein are BNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., 2001; Braasch et al., 2001; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) BNA is used for the bicyclic moiety; in the case of an ethylene group in this position, the term ethyleneoxy (4′-CH₂CH₂—O-2′) BNA is used (Singh et al., 1998; Morita et al., 2003). Methyleneoxy (4′-CH₂—O-2′) BNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., 2000).

An isomer of methyleneoxy (4′-CH₂—O-2′) BNA that has also been discussed is α-L-methyleneoxy (4′-CH₂—O-2′) BNA which has been shown to have superior stability against a 3′-exonuclease. The α-L-methyleneoxy (4′-CH₂—O-2′) BNA's were incorporated into antisense gapmers and chimeras that showed potent antisense activity (Frieden et al., 2003).

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., 1998). BNAs and preparation thereof are also described in WO 98/39352 and WO 99/14226.

Analogs of methyleneoxy (4′-CH₂—O-2′) BNA, phosphorothioate-methyleneoxy (4′-CH₂—O-2′) BNA and 2′-thio-BNAs, have also been prepared (Kumar et al., 1998). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., 1998). In addition, 2′-amino- and 2′-methylamino-BNA's have been prepared and the thermal stability of their duplexes with complementary RNA and DNA strands has been previously reported.

Modified sugar moieties are well known and can be used to alter, typically increase, the affinity of oligomers for targets and/or increase nuclease resistance. A representative list of modified sugars includes, but is not limited to, bicyclic modified sugars (BNA's), including methyleneoxy (4′-CH₂—O-2′) BNA and ethyleneoxy (4′-(CH₂)₂—O-2′ bridge) BNA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

The naturally-occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleotide backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

Described herein are linking groups that link monomers (including, but not limited to, modified and unmodified nucleosides and nucleotides) together, thereby forming an oligomeric compound. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Oligomeric compounds having non-phosphorus linking groups are referred to as oligonucleosides. Modified linkages, compared to natural phosphodiester linkages, can be used to alter, typically increase, nuclease resistance of the oligomeric compound. In certain embodiments, linkages having a chiral atom can be prepared a racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

H. Delivery

A variety of methods may be used to deliver oligonucleotides, including antagomirs and mimics, into a target cell. For cells in vitro embodiments, delivery can often be accomplished by direct injection into cells, and delivery can often be enhanced using hydrophobic or cationic carriers. Alternatively, the cells can be permeabilized with a permeabilization and then contacted with the oligonucleotide. The antagomir can be administered to the subject either as a naked oligonucleotide agent, in conjunction with a delivery reagent, or as a recombinant plasmid or viral vector which expresses the oligonucleotide agent.

For cells in situ, several applicable delivery methods are well-established, e.g., Elmen et al. (2008), Akinc et al. (2008); Esau et al. (2006), Krützfeldt et al. (2005). In particular, cationic lipids (see e.g., Hassani et al., 2004) and polymers such as polyethylenimine (see e.g., Urban-Klein, 2005) have been used to facilitate oligonucleotide delivery. Compositions consisting essentially of the oligomer (i.e., the oligomer in a carrier solution without any other active ingredients) can be directly injected into the host (see e.g., Tyler et al., 1999; McMahon et al., 2002). In vivo applications of duplex RNAs are reviewed in Paroo and Corey (2004).

When microinjection is not an option, delivery can be enhanced in some cases by using Lipofectamine™ (Invitrogen, Carlsbad, Calif.). PNA oligomers can be introduced into cells in vitro by complexing them with partially complementary DNA oligonucleotides and cationic lipid. The lipid promotes internalization of the DNA, while the PNA enters as cargo and is subsequently released. Peptides such as penetratin, transportan, Tat peptide, nuclear localization signal (NLS), and others, can be attached to the oligomer to promote cellular uptake (see e.g., Kaihatsu et al., 2003; Kaihatsu et al., 2004). Alternatively, the cells can be permeabilized with a permeabilization agent such as lysolecithin, and then contacted with the oligomer.

Alternatively, certain single-stranded oligonucleotide agents featured in the instant invention can be expressed within cells from eukaryotic promoters (e.g., Izant and Weintraub, 1985; McGarry and Lindquist, 1986; Scanlon et al., 1991; Kashani-Sabet et al., 1992; Weerasinghe et al., 1991; Ojwang et al., 1992; Chen et al., 1992; Sarver et al., 1990; Thompson et al., 1995). Those skilled in the art realize that any nucleic acid can be expressed in eukaryotic cells from the appropriate DNA/RNA vector. The activity of such nucleic acids can be augmented by their release from the primary transcript by a enzymatic nucleic acid (PCT WO 93/23569; PCT WO 94/02595; Ohkawa et al., 1992; Taira et al., 1991; Ventura et al., 1993; Chowrira et al., 1994).

The recombinant vectors can be DNA plasmids or viral vectors. Oligonucleotide agent-expressing viral vectors can be constructed based on, but not limited to, adeno-associated virus, retrovirus, adenovirus, or alphavirus. In another embodiment, pol III based constructs are used to express nucleic acid molecules of the invention (see for example Morris et al., 2004; U.S. Pat. Nos. 5,902,880 and 6,146,886). The recombinant vectors capable of expressing the oligonucleotide agents can be delivered as described above, and can persist in target cells. Alternatively, viral vectors can be used that provide for transient expression of nucleic acid molecules. Such vectors can be repeatedly administered as necessary. Once expressed, the antagomir interacts with the target RNA (e.g., miRNA or pre-miRNA) and inhibits miRNA activity. In a particular embodiment, the antagomir forms a duplex with the target miRNA, which prevents the miRNA from binding to its target mRNA, which results in increased translation of the target mRNA. Delivery of oligonucleotide agent-expressing vectors can be systemic, such as by intravenous or intra-muscular administration, by administration to target cells ex-planted from a subject followed by reintroduction into the subject, or by any other means that would allow for introduction into the desired target cell (see Couture et al., 1996).

Methods for the delivery of nucleic acid molecules are also described in Akhtar et al. (1992), Akhtar (1995), Maurer et al. (1999), Hofland and Huang (1999), Lee et al. (2000), all of which are incorporated herein by reference. U.S. Pat. No. 6,395,713 and PCT WO 94/02595 and WO 00/53722 further describe general methods for delivery of nucleic acid molecules.

### III. Nucleic Acid Vectors

As mentioned above, miRNAs of the present invention may be delivered and produced via a recombinant vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found.

Vectors include plasmids, cosmids, viruses (bacteriophage, animal viruses, and plant viruses), and artificial chromosomes (e.g., YACs). One of skill in the art would be well equipped to construct a vector through standard recombinant techniques (Sambrook et al., 1989; Ausubel et al., 1996, both incorporated herein by reference). A vector may encode non-modified polypeptide sequences such as a tag or targetting molecule. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A targetting molecule is one that directs the modified polypeptide to a particular organ, tissue, cell, or other location in a subject's body.

A. Expression Vectors or Expression Cassettes

The term “expression vector” refers to a vector containing a nucleic acid sequence coding for at least part of a gene product capable of being transcribed. In some cases, RNA molecules are then translated into a protein, polypeptide, or peptide. In other cases, such as with miRNAs, these sequences are not translated. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it may be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. The identity of tissue-specific promoters or elements, as well as assays to characterize their activity, is well known to those of skill in the art.

2. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

3. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

4. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

5. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

B. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

C. Viral Vectors

There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

### IV. Detection Methods

In some embodiments, it may prove useful to assess the expression of Six1 in a cell from a subject having or suspected of having cancer. In others, miRNAs will be detected. Thus, it is within the general scope of the present invention to provide methods for the detection of proteins and nucleic acids (e.g., mRNAs and miRNAs). Any method of detection known to one of skill in the art falls within the general scope of the present invention.

In particular, nucleic acids can used be as probes or primers for embodiments involving nucleic acid hybridization. As such, they may be used to assess miRNA expression. Commerically available systems, such as Qiagen's miScript System™ are available for detection of miRNAs. Various aspects of nucleic acid detection as discussed below.

A. Protein Detection

1. Immunodetection Methods

As discussed, in some embodiments, the present invention concerns immunodetection methods for binding, purifying, removing, quantifying and/or otherwise detecting biological components such as antigenic regions on polypeptides and peptides. The immunodetection methods of the present invention can be used to identify antigenic regions of a peptide, polypeptide, or protein that has therapeutic implications, particularly in reducing the immunogenicity or antigenicity of the peptide, polypeptide, or protein in a target subject.

Immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature, such as, e.g., Doolittle and Ben-Zeev (1999); Gulbis et al. (1993); De Jager et al. (1993); and Nakamura et al. (1987), each incorporated herein by reference.

In general, the immunobinding methods include obtaining a sample suspected of containing a protein, polypeptide and/or peptide, and contacting the sample with a first antibody, monoclonal or polyclonal, in accordance with the present invention, as the case may be, under conditions effective to allow the formation of immunocomplexes.

These methods include methods for purifying a protein, polypeptide and/or peptide from organelle, cell, tissue or organism's samples. In these instances, the antibody removes the antigenic protein, polypeptide and/or peptide component from a sample. The antibody will preferably be linked to a solid support, such as in the form of a column matrix, and the sample suspected of containing the protein, polypeptide and/or peptide antigenic component will be applied to the immobilized antibody. The unwanted components will be washed from the column, leaving the antigen immunocomplexed to the immobilized antibody to be eluted.

The immunobinding methods also include methods for detecting and quantifying the amount of an antigen component in a sample and the detection and quantification of any immune complexes formed during the binding process. Here, one would obtain a sample suspected of containing an antigen or antigenic domain, and contact the sample with an antibody against the antigen or antigenic domain, and then detect and quantify the amount of immune complexes formed under the specific conditions.

In terms of antigen detection, the biological sample analyzed may be any sample that is suspected of containing an antigen or antigenic domain, such as, for example, a tissue section or specimen, a homogenized tissue extract, a cell, an organelle, separated and/or purified forms of any of the above antigen-containing compositions, or even any biological fluid that comes into contact with the cell or tissue, including blood and/or serum.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

2. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, antibodies are immobilized onto a selected surface exhibiting protein affinity, such as a well in a polystyrene microtiter plate. Then, a test composition suspected of containing the antigen, such as a clinical sample, is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with antibodies. After binding and/or washing to remove non-specifically bound immune complexes, the bound anti-antibodies are detected. Where the initial antibodies are linked to a detectable label, the immune complexes may be detected directly. Again, the immune complexes may be detected using a second antibody that has binding affinity for the first antibody, with the second antibody being linked to a detectable label.

Another ELISA in which the antigens are immobilized, involves the use of antibody competition in the detection. In this ELISA, labeled antibodies against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

3. Immunohistochemistry

The antibodies of the present invention may also be used in conjunction with both fresh-frozen and/or formalin-fixed, paraffin-embedded tissue blocks prepared for study by immunohistochemistry (IHC). For example, immunohistochemistry may be utilized to characterize Six1 or to evaluate the amount Six1 in a cell. The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Immunohistochemistry or IHC refers to the process of localizing proteins in cells of a tissue section exploiting the principle of antibodies binding specifically to antigens in biological tissues. It takes its name from the roots “immuno,” in reference to antibodies used in the procedure, and “histo,” meaning tissue. Immunohistochemical staining is widely used in the diagnosis and treatment of cancer. Specific molecular markers are characteristic of particular cancer types.

Visualizing an antibody-antigen interaction can be accomplished in a number of ways. In the most common instance, an antibody is conjugated to an enzyme, such as peroxidase, that can catalyse a color-producing reaction. Alternatively, the antibody can also be tagged to a fluorophore, such as FITC, rhodamine, Texas Red, Alexa Fluor, or DyLight Fluor. The latter method is of great use in confocal laser scanning microscopy, which is highly sensitive and can also be used to visualize interactions between multiple proteins.

Briefly, frozen-sections may be prepared by rehydrating 50 mg of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

There are two strategies used for the immunohistochemical detection of antigens in tissue, the direct method and the indirect method. In both cases, the tissue is treated to rupture the membranes, usually by using a kind of detergent called Triton X-100.

The direct method is a one-step staining method, and involves a labeled antibody (e.g. FITC conjugated antiserum) reacting directly with the antigen in tissue sections. This technique utilizes only one antibody and the procedure is therefore simple and rapid. However, it can suffer problems with sensitivity due to little signal amplification and is in less common use than indirect methods.

The indirect method involves an unlabeled primary antibody (first layer) which reacts with tissue antigen, and a labeled secondary antibody (second layer) which reacts with the primary antibody. The secondary antibody must be against the IgG of the animal species in which the primary antibody has been raised. This method is more sensitive due to signal amplification through several secondary antibody reactions with different antigenic sites on the primary antibody. The second layer antibody can be labeled with a fluorescent dye or an enzyme.

In a common procedure, a biotinylated secondary antibody is coupled with streptavidin-horseradish peroxidase. This is reacted with 3,3′-Diaminobenzidine (DAB) to produce a brown staining wherever primary and secondary antibodies are attached in a process known as DAB staining. The reaction can be enhanced using nickel, producing a deep purple/gray staining.

The indirect method, aside from its greater sensitivity, also has the advantage that only a relatively small number of standard conjugated (labeled) secondary antibodies needs to be generated. For example, a labeled secondary antibody raised against rabbit IgG, which can be purchased “off the shelf,” is useful with any primary antibody raised in rabbit. With the direct method, it would be necessary to make custom labeled antibodies against every antigen of interest.

4. Protein Arrays

Protein array technology is discussed in detail in Pandey and Mann (2000) and MacBeath and Schreiber (2000), each of which is herein specifically incorporated by reference. These arrays, typically contain thousands of different proteins or antibodies spotted onto glass slides or immobilized in tiny wells, allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds. In certain embodiments such technology can be used to quantitate a number of proteins in a sample, such as Six1.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

B. Nucleic Acid Detection

Nucleic acids can used be as probes or primers for embodiments involving nucleic acid hybridization. As such, they may be used to assess mRNA expression for Six1. Various aspects of nucleic acid detection as discussed below.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In particular embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

2. In Situ Hybridization

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g. plant seeds, Drosophila embryos), in the entire tissue (whole mount ISH). This is distinct from immunohistochemistry, which localizes proteins in tissue sections. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

3. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al. (1988), each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific mRNA species isolated from a cell, such as a Six1 or Eya-encoding transcript. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100-fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Chip Technologies and Nucleic Acid Arrays

The present invention may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### V. Methods of Therapy

In some embodiments, the invention provides compositions and methods for the treatment of cancer. In one embodiment, the invention provides a method of treating cancer comprising administering to a patient an effective amount of a Six1 miRNA. This treatment may be further combined with additional cancer treatments. One of skill in the art will be aware of many treatments that may be combined with the methods of the present invention, some but not all of which are described below.

In general, the cancers will be characterized by overexpression of Six1. Thus, it is contemplated that a wide variety of tumors may be treated using Six1 miRNAs, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

A. miRNAs

One therapy approach is the provision, to a subject, of a Six1 miRNA. The miRNA is generally produced by an automated synthesizer, although it may also be produced recombinantly. Formulations for delivery of the miRNA are selected based on the route of administration and purpose including, but not limited to, liposomal formulations and classic pharmaceutical preparations.

B. Genetic Delivery

The inventors also contemplate the use of expression constructs encoding miRNAs. The construction and structure of viral vectors is discussed above.

Administration protocols would generally involve intratumoral, local or regional (to the tumor) administration, as well as systemic administration in appropriate clinical situations.

C. Formulations and Routes for Administration to Patients

In some embodiments, the invention provides a method of treating cancer comprising providing to a patient an effective amount of a Six1 miRNA. Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the invention, methods include identifying a patient in need of treatment. A patient may be identified, for example, based on taking a patient history or based on findings on clinical examination.

D. Cancer Combination Treatments

In some embodiments, the method further comprises treating a patient with cancer with a conventional cancer treatment. One goal of current cancer research is to find ways to improve the efficacy of chemo- and radiotherapy, such as by combining traditional therapies with other anti-cancer treatments. In the context of the present invention, it is contemplated that this treatment could be, but is not limited to, chemotherapeutic, radiation, a polypeptide inducer of apoptosis or other therapeutic intervention. It also is conceivable that more than one administration of the treatment will be desired.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gen silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Gene Therapy

In yet another embodiment, the secondary treatment is a gene therapy in which a therapeutic polynucleotide is administered before, after, or at the same time as a Six1 or Eya inhibitor is administered. Delivery of a Six1 or Eya inhibitor in conjunction with a vector encoding one of the following gene products may have a combined anti-hyperproliferative effect on target tissues. A variety of proteins are encompassed within the invention, some of which are described below.

a. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA or siRNA directed to a particular inducer of cellular proliferation is used to prevent expression of the inducer of cellular proliferation.

The proteins FMS and ErbA are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

b. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, mda-7, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p′73, VHL, MMAC1/Six1 or Eya2, DBCCR-1, FCC, rsk-3, p2′7, p27/p16 fusions, p21/p27 fusions, anti-thrombotic genes (e.g., COX-1, TFPI), PGS, Dp, E2F, ras, myc, neu, raf, erb, fins, trk, ret, gsp, hst, abl, E1A, p300, genes involved in angiogenesis (e.g., VEGF, FGF, thrombospondin, BAI-1, GDAIF, or their receptors) and MCC.

c. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

d. Antagomirs of Endogenous miRNAs that Upregulate Six1

As discussed above, it is possible that miRNAs can directly or indirectly lead to an increase in Six1 levels (for example, the miRNA targets a repressor of Six1). In one aspect, the invention would relate to delivery of antagomirs of miRNAs.

6. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon α, β, and γ; IL-2 and other cytokines; F42K and other cytokine analogs; or MIP-1, MIP-1β, MCP-1, RANTES, and other chemokines It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

E. Dosage

An miRNA can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of antagomir (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of antagomir per kg of bodyweight. The unit dose, for example, can be administered by injection (e.g., intravenous or intramuscular, intrathecally, intratumorally or directly into an organ), inhalation, or a topical application.

Delivery of an miRNA directly to an organ can be at a dosage on the order of about 0.00001 mg to about 3 mg per organ, or particularly about 0.0001-0.001 mg per organ, about 0.03-3.0 mg per organ, about 0.1-3.0 mg per organ or about 0.3-3.0 mg per organ.

Significant modulation of target gene expression may be achieved using nanomolar/submicromolar or picomolar/subnamomolar concentrations of the oligonucleotide, and it is typical to use the lowest concentration possible to achieve the desired resultant increased synthesis, e.g., oligonucleotide concentrations in the 1-100 nM range are contemplated; more particularly, the concentration is in the 1-50 nM, 1-25 nM, 1-10 nM, or picomolar range. In particular embodiments, the contacting step is implemented by contacting the cell with a composition consisting essentially of the oligonucleotide.

In one embodiment, the unit dose is administered once a day, e.g., or less frequently less than or at about every 2, 4, 8 or 30 days. In another embodiment, the unit dose is not administered with a frequency (e.g., not a regular frequency). For example, the unit dose may be administered a single time. Because oligonucleotide agent can persist for several days after administering, in many instances, it is possible to administer the composition with a frequency of less than once per day, or, for some instances, only once for the entire therapeutic regimen.

An miRNA featured in the invention can be administered in a single dose or in multiple doses. Where the administration of the miRNA is by infusion, the infusion can be a single sustained dose or can be delivered by multiple infusions. Injection of the miRNA can be directly into the tissue at or near the site of interest. Multiple injections of can be made into the tissue at or near the site.

In a particular dosage regimen, the miRNA is injected at or near a disease site once a day for seven days, for example, into a tumor, a tumor bed, or tumor vasculature. Where a dosage regimen comprises multiple administrations, it is understood that the effective amount of the miRNA administered to the subject can include the total amount of miRNA administered over the entire dosage regimen. One skilled in the art will appreciate that the exact individual dosages may be adjusted somewhat depending on a variety of factors, including the specific antagomir being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration. Variations in these dosage levels can be adjusted using standard empirical routines of optimization, which are well-known in the art. The precise therapeutically effective dosage levels and patterns can be determined by the attending physician in consideration of the above-identified factors.

In one embodiment, a subject is administered an initial dose, and one or more maintenance doses of an miRNA. The maintenance dose or doses are generally lower than the initial dose, e.g., one-half less of the initial dose. The maintenance doses are generally administered no more than once every 5, 10, or 30 days. Further, the treatment regimen may last for a period of time which will vary depending upon the nature of the particular disease, its severity and the overall condition of the patient. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

Certain factors may influence the dosage required to effectively treat a subject, including but not limited to the severity of the disease or disorder, previous treatments, the general health and/or age of the subject, and other diseases present. It will also be appreciated that the effective dosage of the antagomir used for treatment may increase or decrease over the course of a particular treatment. Changes in dosage may result and become apparent from the results of diagnostic assays. For example, the subject can be monitored after administering an antagomir composition. Based on information from the monitoring, an additional amount of the antagomir composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC₅₀'s found to be effective in in vitro and in vivo animal models.

### VI. Examples

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Luciferase Assays. psi-CHECK2 dual luciferase vector (Promega) was used for luciferase readouts. The 3′UTR of Six1 was obtained by PCR from MCF7 cDNA and engineered with restriction sites to clone into the multiple cloning site of the psi-Check2 vector, which sits after the SV40 driven Renilla luciferase gene and before a synthetic polyA site. The firefly luciferase gene exists on the same plasmid driven by a separate promoter (HSV-TK), which is used for normalization of 3′UTR studies. HEK293 cells were co-transfected in 24-well plates with 50 ng Six1 3′UTR-luc vector and 20 nm of selected miRNA mimics (Dharmacon). Six1-Luc vectors were mutated at selected miRNA seed match regions using site-directed mutagenesis (Stratagene), and transfected at 50 ng in same manner. Transfections were conducted using Lipofectamine 2000 reagent (Invitrogen). Firefly and Renilla luciferase activities were measured consecutively by using the Dual Luciferase Assay (Promega) 48 hours after transfection. Each sample was assayed in triplicate.

qRT-PCR. RNA for quantitative Real-Time PCR was extracted from cells using the miRNeasy mini kit for RNA from Qiagen. cDNA was generated from RNA isolations using Superscript III RT-PCR kit (Invitrogen) for mRNA detection using 1 μg of total RNA per reaction. For miRNA assays, cDNA was prepared from the miScript RT Reaction kit (Qiagen). All cDNA was diluted 1:2 for qPCR assays. miRNAs were assayed using the miScript Primer Assays from Qiagen, which recognize the specific mature miRNAs. Syber green master mix was also purchased from Qiagen. miRNA samples were normalized to RNU6B (Qiagen) and Six1 (ABI, taqman gene expression assays) mRNA levels were normalized to Cyclophililn B (ABI, taqman gene expression assays). Gene expression levels were quantified using the BioRad CFX96 Sequence detection system (BioRad). Comparative real-time PCR was performed in triplicate, including no-template controls. Relative expression was calculated using the comparative Ct method (ddCT).

Western Blotting. Protein extractions were performed with RIPA buffer to obtain whole cell lysates from BT549 cells after treatment with miRNA mimics. BT549 cells were seeded into 6-well plates and transfected with 20 nm of miRNA mimics separately (Neg, miR-185, miR-639, and miR-571) or all together (miR-185+639+571) versus a negative control mimic. Protein concentrations were determined using the Lowry Dc Protein Assay (BioRad), and 50 μg of protein of each lysate was loaded onto a 12% SDS-PAGE gel. Protein blotting was performed with the lab supply of Six1 polyclonal Antibody (Ford2), and anti-B-Tubulin antibody for loading control (Sigma).

Cell culture: MCF7 cells were cultured in DMEM 10% FBS, 1% L-glut, 0.1% PenStrep. MCF7-CTRL cells were previously stably transfected with the CAT gene while MCF7-Six were stably transfected with Six1. We used 3 separate clonal isolates of each condition, MCF7-Six1 and MCF7-CTRL. Six1 siRNA, smartPOOL purchased from Dharmacon, were transfected using Lifofectamine protocol (Invitrogen). Cells were incubated at 37° C. for 48 hours after transfection and RNA was collected for analysis.

miRNA microarray: RNA from MCF7-Six1 and MCF7-CTRL cells were submitted to Thermo Fisher for analysis using a HT-HgU133A Affymetrix chip. Results were analyzed by Thermo Fisher.

### Example 2

### Results

Compiled candidate miRNAs for Six1 repression. To determine possible miRNAs that may regulate Six1, we used publically available prediction websites which predict miRNA targets based on different algorithyms for seed match complementarity in the 3′UTR of target messages. A list of predicted miRNAs from the databases Pictar, Targetscan, and miRBase were compiled, and the strongest predicted miRNAs between TargetScan and Pictar were chosen (FIG. 1).

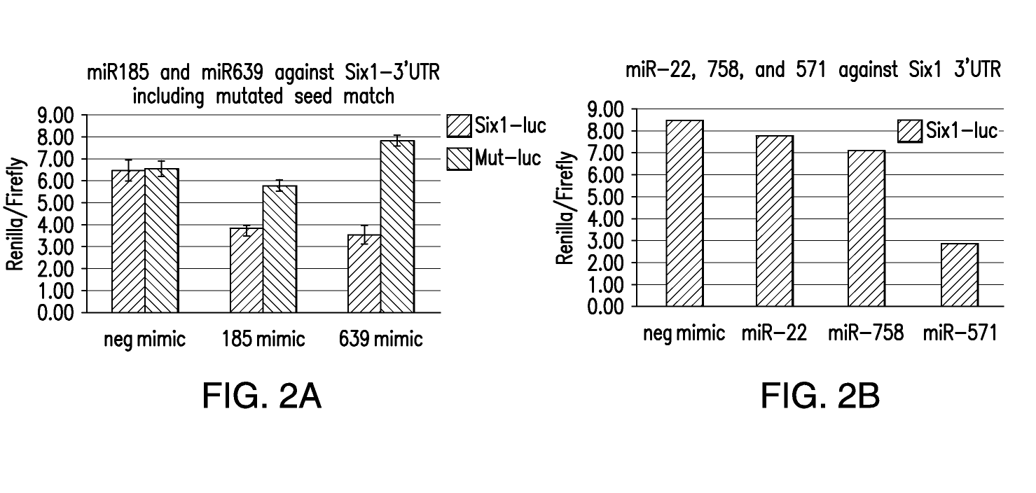

miR-185, miR-639, and miR-571 can target Six1 3′UTR. To determine if the predicted miRNAs can directly repress Six1, the inventors constructed a luciferase vector containing the 3′UTR of Six1 and co-transfected selected miRNA mimics with the luciferase vector. Of the predicted miRNAs to regulate Six1, 3 of the 5 tested showed a repression of Renilla luciferase on the 3′UTR of Six1 in a luciferase vector (FIGS. 2A-B). To confirm specific targeting of these miRNA to Six1-3′UTR, the specific seed match regions of miR-185 and miR-639 were mutated using site directed mutagenesis (FIG. 2A). Luciferase readouts with the mutated seed regions showed a de-repression of Renilla luciferase normalized to firefly when co-transfected with selected miRNA mimics. As of yet, no mutated seed region for miR-571 has been constructed, although this remains to be tested.

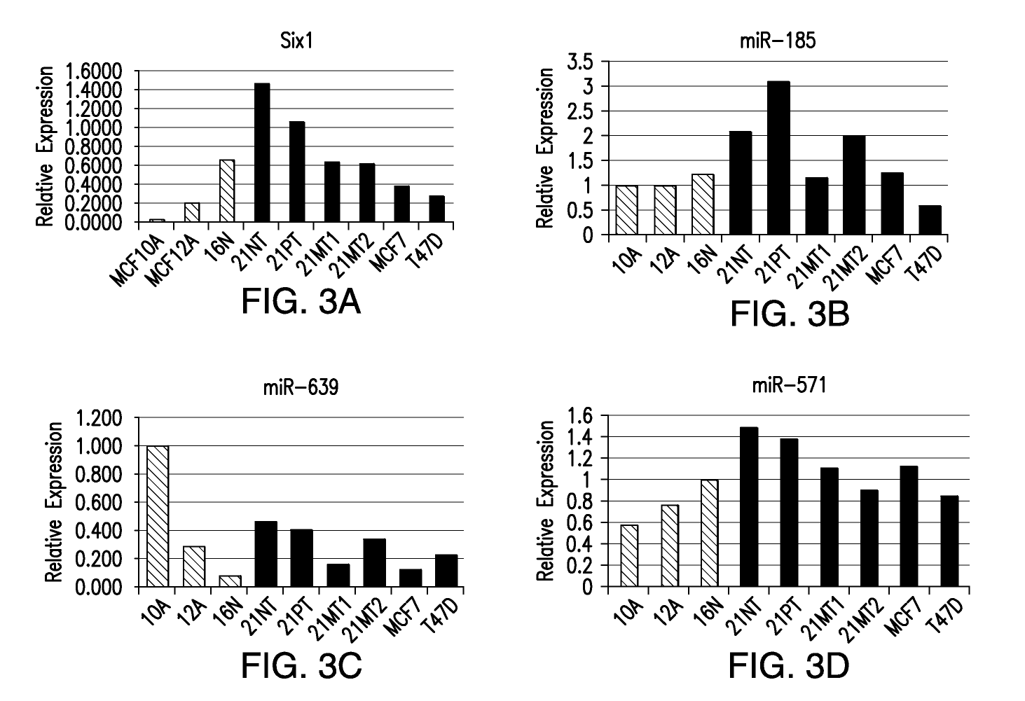

Selected miRNAs show no definitive correlation among normal vs. breast cancer cell lines. To determine if miR185, 639, and 571 have differential expression between normal and breast cancer cell lines, the inventors extracted RNA from 3 normal breast cell lines (MCF10A, MCF12A, and 16N) along with 6 breast cancer cell lines (21NT, 21PT, 21MT1, 21MT2, MCF7, and T47D). RNA was processed into cDNA and run on qPCR to measure the differential expression of Six1 mRNA levels versus the levels of miRNA in these cell lines. As expected, the breast cancer cell lines are shown to have a higher expression of Six1 (FIG. 3A), however, the expression of miR-185, -571, and -639 do not show a stong correlation with endogenous Six1 levels in these cell lines (FIGS. 3B-D). One would expect these miRNAs to have low expression in cell lines with high Six1 levels, and we do not see this correlation in these cell lines. However, the inventors hope to look at levels of these miRNA in breast cancer tissues by ISH where they may be able to see a more accurate display of miRNA expression versus Six1 expression.

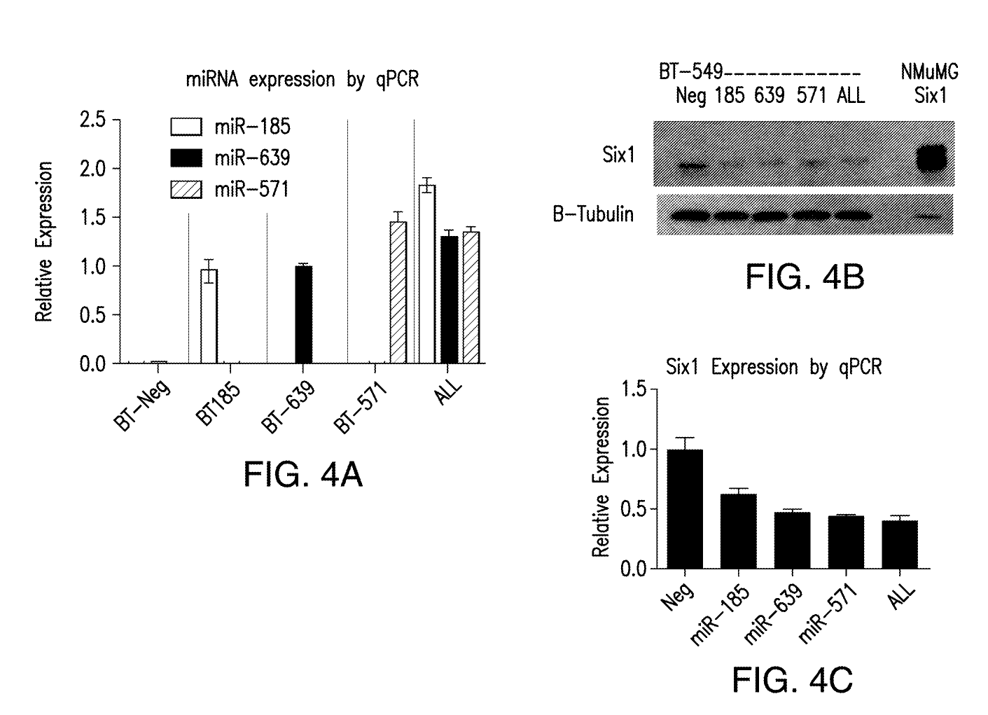

miR-185, miR-639, and miR-571 Repress Endogenous Six1 Protein Expression. The selected miRNAs which show repression of Six1 3′UTR in Luciferase experiments were next tested to see if they have the ability to repress endogenous levels of Six1 protein. The inventors chose the breast cancer cell line, BT549, because of its high endogenous expression of Six1, so that they would be able to view any change in protein levels by western blot. miRNA mimics for miR-185, -639, and -571 were transfected into BT549 cells, along with a negative control mimic. Additionally, all three miRNA were added together to see if there is any coorperativity in repression of Six1. Protein was extracted after 48 hours and run on a western blot to probe for Six1 expression. From this blot, the inventors see that all three miRNA do indeed repress Six1 at the protein level (FIG. 4B). Additionally, RNA was also extracted from this experiment to test for Six1 levels on the mRNA level, which also shows a repression in the presence of selected miRNA mimics (FIG. 4C). The addition of all three miRNA together did not seem to have a cooperative effect on repression of Six1 protein level (FIG. 4B).

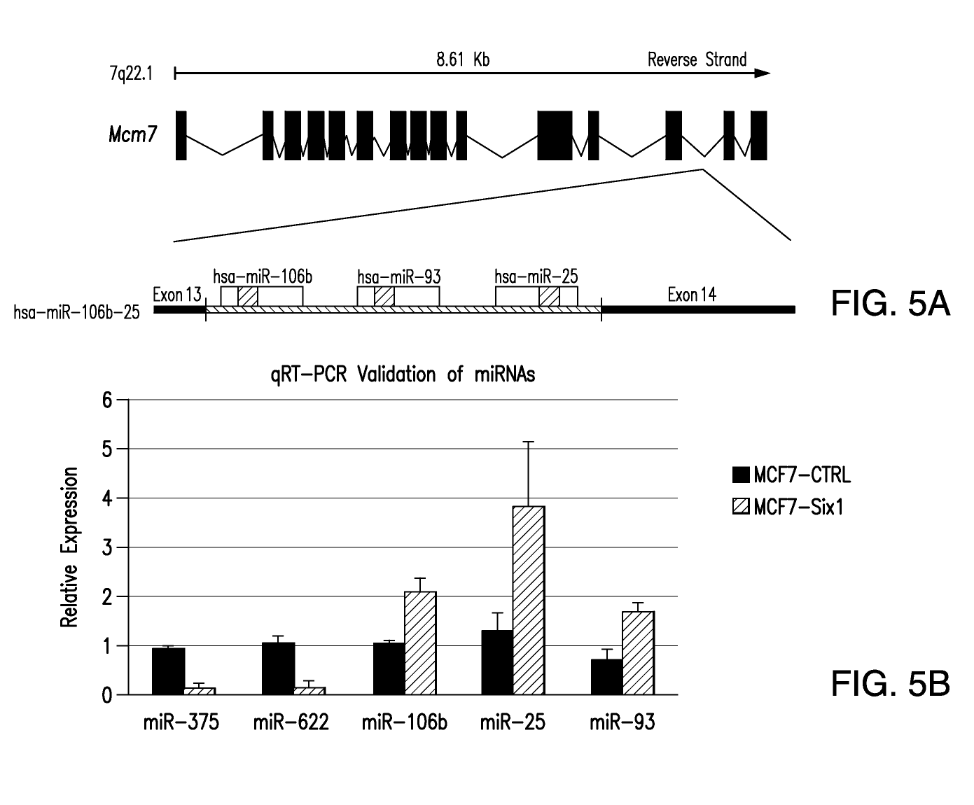

A microRNA microarray reveals promising miRNAs controlled by Six1. RNA from MCF-CTRL and MCF7-Six1 transfected cells were submitted for a platform miRNA microarray analysis. miRNAs in the analysis which contained a Q-value lower than 0.05 were selected for further investigation (Table 1). Also in the analysis, which did not make the chart, was miR-375 which was present in 10 of the 12 MCF7-CTRL replicates and none of the 12 MCF7-Six1 replicates. This miR does not have a Q value, but was added to the list of miRNAs to investigate. To further confirm that these miRNAs were significantly changed between MCF7-CTRL and MCF7-Six1 we validated each one by qRT-PCR (FIG. 5B). From this qRT-PCR validation, the inventors found 4 miRNAs which were significantly changed in expression between MCF7-CTRL and MCF7-Six1. These miRNAs were hsa-miR-622 and hsa-miR-375, which are downregulated upon Six1 expression, and hsa-miR-106b and hsa-miR-25, which are upregulated upon Six1 expression (FIG. 5B).

Interestingly, the inventors found that miRNAs miR-106b and miR-25 sit in a cluster with another miRNA, hsa-miR-93, referred to as the miR106b-25 cluster. This cluster is located in a 515-bp region at Ch7q22, in intron 13 of the MCMI gene (FIG. 5A). This miRNA was not present on the original microarray screen, therefore the inventors also wanted to include it in these validation studies to see if Six1 may regulate this entire cluster of miRNAs. Indeed, they do see upregulation of miR-93 along with the other two miRNAs in this cluster (FIG. 5B).

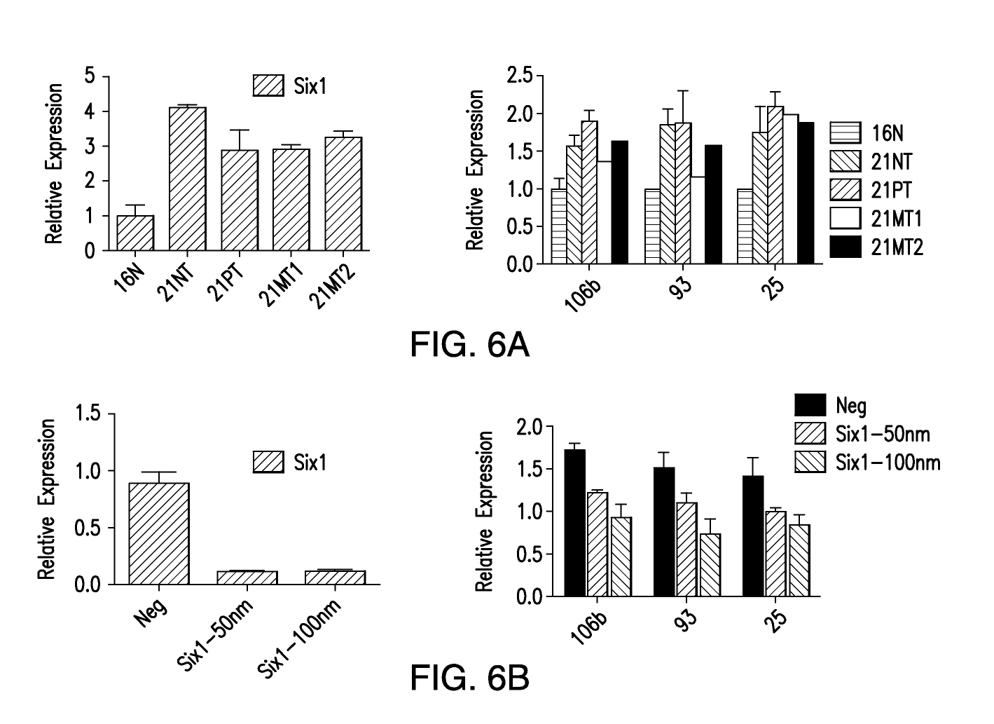

The miR106b-25 cluster follows endogenous Six1 regulation. The inventors used the 21T series cell lines to look at endogenous Six1 expression. This is breast cancer isogenic cell line series, and consists of 16N, which is from normal breast tissue, 21PT and 21NT, which are from the primary breast tumor, and 21MT1 and 21MT2, which are from the metastatic tumor site. The carcinogenic cells in this line (21PT, 21NT, 21MT1, and 21MT2) all express high Six1 as compared to the normal breast cell line 16N (FIG. 6A). Along with this, the inventors also discovered that each of the miRNAs in the miR106b-25 cluster also are upregulated in all the carcinogenic lines as compared to 16N (FIG. 6A), thus following the inventors' finding that Six1 upregulates this cluster of miRNAs.

Additionally, the inventors used the 21PT cell line to see if the miR106b-25 cluster would follow Six1 expression if Six1 was knocked down in these cells. They knocked down Six1 by siRNA in the 21PT cells, and after confirmation of the Six1 knock down, and then tested the same samples on qRT-PCR for expression of the miR106b-25 miRNAs. The inventors found that this miRNA cluster was also down regulated in expression following the Six1 siRNA, suggesting that the miR106b-25 cluster is truly regulated by Six1. The inventors hope to further investigate if this is a direct or indirect regulation.

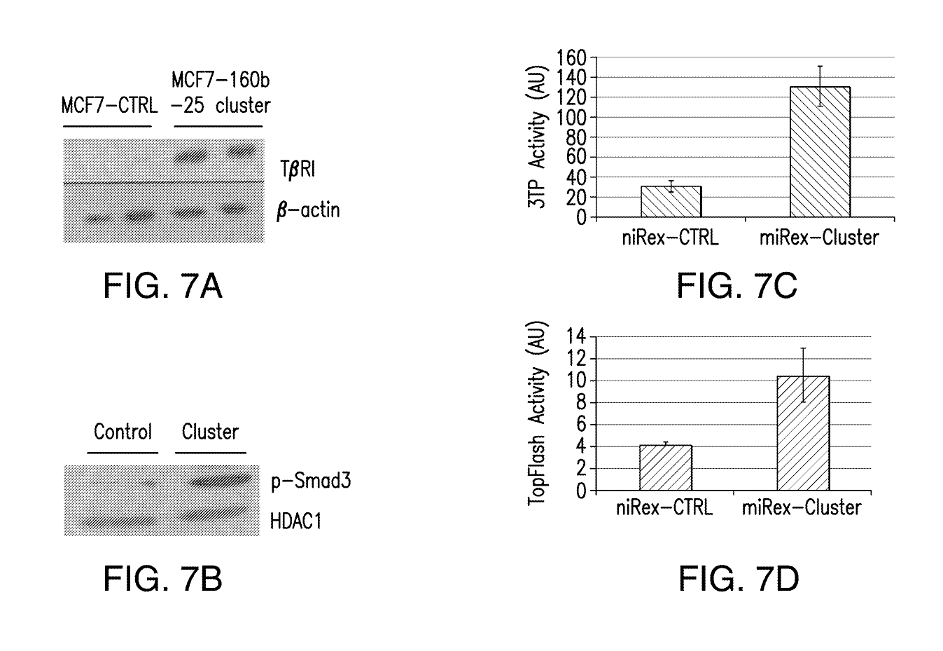

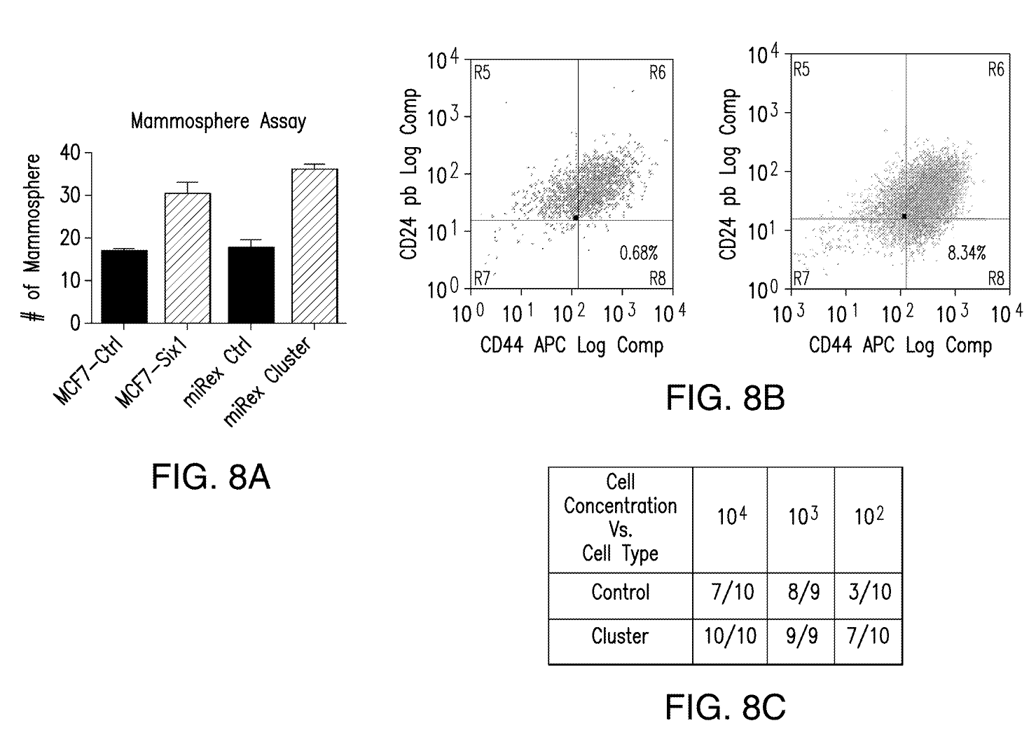

Downstream miRNA data. Interestingly, the inventors observed that if they express the miR-106b-25 cluster alone in MCF7 cells, they can also affect the TGFβ signaling pathway similar to Six1 overexpression. They observe an increase in TBR-I protein as well as an increase in phosphorylated Smad3 in the cluster overexpressing MCF7 cells (FIGS. 7A-B). Also, with transient overexpression of this cluster, they also see an increase in TGFβa transcriptional activity (3TP activity) and β-catenin transcriptional activity (Topflash) (FIGS. 7C-D). Lastly, they also observe a cancer stem cell phenotype with overexpression of the miR106b-25 cluster. This phenotype is representative of what is present in Six1 overexpressing conditions, where the inventors see an increase in mammosphere formation capability (FIG. 8A). Additionally, cluster overexpressing cells exhibit an increase in the stem cell population (CD44 low/CD24 hi) by flow cytometry (FIG. 8B), and an increase in tumor formation at low concentrations of cells injected into the mammary fat pad of mice (FIG. 8C), suggesting that the miR106b-25 cluster can recapitulate many of the stem cell phenotypes that the inventors observe with just Six1 overexpression.

TABLE 1
miRNA microarray analysis between MCF7-Cat control vs. MCF7-Six1 RNA

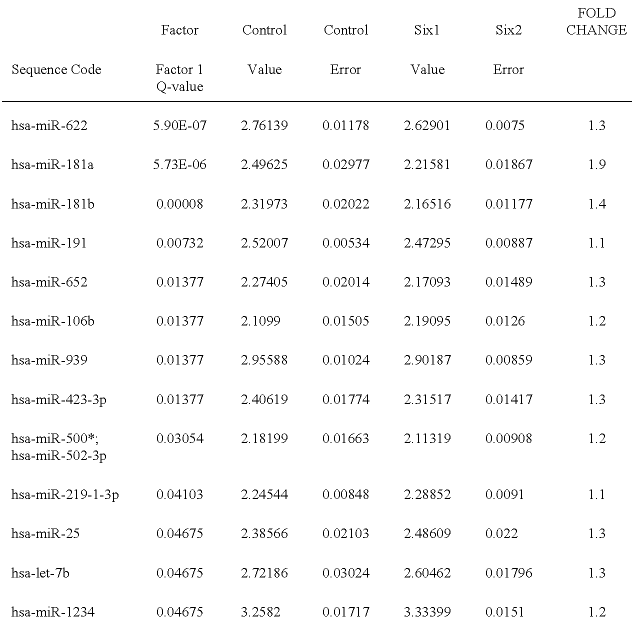

The top 13 miRNAs from the microarray were selected by having a Q-value less than 0.05. The -fold change between MCF7-Cat and MCF7-Six1 is shown in the last column. The highlighted rows show the promising miRNAs which were confirmed by qRT-PCR. Not included on this table is an additional miRNA which was completely absent in MCF7-Six1 and present in 10 of 12 replicates in MCF7-Cat, hsa-miR-375.

All of the methods and apparatus disclosed and claimed herein can be made and executed without undue experimentation in light of the present disclosure. While the compositions and methods of this invention have been described in terms of particular embodiments, it will be apparent to those of skill in the art that variations may be applied to the methods and apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,683,195
U.S. Pat. No. 4,683,202
U.S. Pat. No. 4,683,202
U.S. Pat. No. 4,800,159
U.S. Pat. No. 4,879,236
U.S. Pat. No. 4,883,750
U.S. Pat. No. 4,981,957
U.S. Pat. No. 5,118,800
U.S. Pat. No. 5,143,854
U.S. Pat. No. 5,202,231
U.S. Pat. No. 5,242,974
U.S. Pat. No. 5,288,644
U.S. Pat. No. 5,319,080
U.S. Pat. No. 5,324,633
U.S. Pat. No. 5,359,044
U.S. Pat. No. 5,384,261
U.S. Pat. No. 5,393,878
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,786
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,514,785
U.S. Pat. No. 5,519,134
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,556,752
U.S. Pat. No. 5,561,071
U.S. Pat. No. 5,567,811
U.S. Pat. No. 5,571,639
U.S. Pat. No. 5,576,427
U.S. Pat. No. 5,580,726
U.S. Pat. No. 5,580,732
U.S. Pat. No. 5,591,722
U.S. Pat. No. 5,593,839
U.S. Pat. No. 5,597,909
U.S. Pat. No. 5,599,672
U.S. Pat. No. 5,599,695
U.S. Pat. No. 5,610,287
U.S. Pat. No. 5,610,300
U.S. Pat. No. 5,624,711
U.S. Pat. No. 5,627,053
U.S. Pat. No. 5,631,134
U.S. Pat. No. 5,639,603
U.S. Pat. No. 5,639,873
U.S. Pat. No. 5,646,265
U.S. Pat. No. 5,654,413
U.S. Pat. No. 5,658,734
U.S. Pat. No. 5,658,873
U.S. Pat. No. 5,661,028
U.S. Pat. No. 5,665,547
U.S. Pat. No. 5,667,972
U.S. Pat. No. 5,670,633
U.S. Pat. No. 5,695,940
U.S. Pat. No. 5,700,637
U.S. Pat. No. 5,700,920
U.S. Pat. No. 5,739,169
U.S. Pat. No. 5,744,305
U.S. Pat. No. 5,792,747
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,871,986
U.S. Pat. No. 5,876,932
U.S. Pat. No. 5,882,864
U.S. Pat. No. 5,900,481
U.S. Pat. No. 5,902,880
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 6,004,755
U.S. Pat. No. 6,087,102
U.S. Pat. No. 6,146,886
U.S. Pat. No. 6,268,490
U.S. Pat. No. 6,368,799
U.S. Pat. No. 6,383,749
U.S. Pat. No. 6,395,713
U.S. Pat. No. 6,525,191
U.S. Pat. No. 6,531,584
U.S. Pat. No. 6,600,032
U.S. Pat. No. 6,617,112
U.S. Pat. No. 6,638,717
U.S. Pat. No. 6,670,461
U.S. Pat. No. 6,720,138
U.S. Pat. No. 6,770,748
U.S. Pat. No. 6,794,499
U.S. Pat. No. 7,034,133
U.S. Pat. No. 7,053,207
U.S. Patent Publn. 2008/0009439
U.S. Patent Pubn. 2003/0082807
U.S. Patent Pubn. 2003/0207841
U.S. Patent Pubn. 2004/0014959
U.S. Patent Pubn. 2004/0143114
U.S. Patent Pubn. 2004/0171570
U.S. Patent Pubn. 2004/0219565
U.S. Patent Pubn. 2007/0213292
U.S. Patent Pubn. 2007/0287831
U.S. Patent Pubn. 2008/0015162
U.S. Patent Pubn. 2008/0032945
Abbondanzo et al., Breast Cancer Res. Treat., 16:182(151), 1990.
Akhtar et al., Trends in Cell Bio., 2:139, 1992.
Akhtar, In: Delivery Strategies for Antisense Oligonucleotide Therapeutics, 1995.
Akinc et al. Nat. Biotechnol., 2008 May; 26(5):561-9, 2008.
Allerson et al., J. Med. Chem., 48:901-904, 2005.
Allred et al., Arch. Surg., 125(1):107-113, 1990.
Ambros et al. RNA, 9:277-279, 2003.
Arap et al., Cancer Res., 55(6):1351-1354, 1995.
Austin-Ward and Villaseca, Revista Medica de Chile, 126(7):838-845, 1998.
Ausubel et al., In: Current Protocols in Molecular Biology, John, Wiley & Sons, Inc, NY, 1994; 1996.
Baichwal and Sugden, In: Gene Transfer, Kucherlapati (Ed.), Plenum Press, NY, 117-148, 1986.
Bakhshi et al., Cell, 41(3):899-906, 1985.
Bellus, J. Macromol. Sci. Pure Appl. Chem., A31(1): 1355-1376, 1994.
Bentin and Nielsen, Biochemistry, 35:8863-8869, 1996.
Braasch et al., Methods, 23(2):97-107, 2001.
Brennecke et al., Cell, 113(1):25-36, 2003.
Brown et al. Immunol. Ser., 53:69-82, 1990.
Bukowski et al., Clinical Cancer Res., 4(10):2337-2347, 1998.
Caldas et al., Cancer Res., 54:3568-3573, 1994.
Calin et al., Proc. Natl. Acad. Sci. USA, 99(24):15524-15529, 2002.
Carbonelli et al., FEMS Microbiol. Lett., 177(1):75-82, 1999.
Carrington and Ambros, Science, 301(5631):336-338, 2003.
Chang et al., J. Exp. Med., 200:1359-1370, 2004.
Chen et al., Fed. of Eur. Biochem Soc., 309:115-118, 1992.
Chen et al., Mol. Microbiol., 53843-856, 2004.
Cheng et al., Cancer Res., 54(21):5547-5551, 1994.
Choung et al., Biochem. Biophys. Res. Commun., 342:919-927, 2006.
Chowrira et al., J. Biol. Chem., 269(41):25856-25864, 1994.
Christodoulides et al., Microbiology, 144(Pt 11):3027-3037, 1998.
Cleary and Sklar, Proc. Natl. Acad. Sci. USA, 82(21):7439-7443, 1985.
Cleary et al., J. Exp. Med., 164(1):315-320, 1986.
Cocea, Biotechniques, 23(5):814-816, 1997.
Coupar et al., Gene, 68:1-10, 1988.
Couture et al., Trends in Genetics, 12:510, 1996.
Davidson et al., J. Immunother., 21(5):389-398, 1998.
De Jager et al., Semin. Nucl. Med., 23(2):165-179, 1993.
Demidov et al., ChemBiochem., 2:133-139, 2001.
Doolittle and Ben-Zeev, Methods Mol. Biol., 109:215-237, 1999.
Egholm et al., Nature, 365(6446):566-568, 1993.
Elayadi et al., Nucleic Acids Res., 29(8):1683-9, 2001.
Elmén et al., Nature, 452(7189):896-9, 2008.
Esau et al. Cell Metab., 3(2):87-98, 2006.
European Appln. 320 308
European Appln. 329 822
European Appln. EP 373 203
European Appln. EP 785 280
European Appln. EP 799 897
Faruqi et al., Proc. Natl. Acad. Sci. USA, 95:1398-1403, 1998.
Fatkin et al., J. Clin. Invest., 106(11):1351-1359, 2000.
Frieden et al., Nucl. Nucleic Acids, 22:1041-1043, 2003.
Friedmann, Science, 244:1275-1281, 1989.
Frohman, In: PCR Protocols: A Guide To Methods And Applications, Academic Press, N.Y., 1990.
GB Appln. 2 202 328
Gottardo et al., Urol. Oncol., 25(5):387-392, 2007.
Gulbis and Galand, Hum. Pathol., 24(12):1271-1285, 1993.
Haeberli et al., Nucleic Acids Res., 33:3965-3975, 2005.
Hall et al., Nucleic Acids Res., 32:5991-6000, 2004.
Hall et al., Nucleic Acids Res., 34:2773-2781, 2006.
Hanibuchi et al., Int. J. Cancer, 78(4):480-485, 1998.
Hassani et al., J. Gene Med., 7(2):198-207, 2005.
Hellstrand et al., Acta Oncologica, 37(4):347-353, 1998.
Hermonat and Muzycska, Proc. Natl. Acad. Sci. USA, 81:6466-6470, 1984.
Hofland and Huang, Handb. Exp. Pharmacol., 137:165, 1999.
Holstege et al., EMBO J., 16:7468-7480, 1997.
Horwich et al. J. Virol., 64:642-650, 1990.
Hoshika et al., Nucleic Acids Res., 32:3815-3825, 2004.
Hui and Hashimoto, Infection Immun., 66(11):5329-5336, 1998.
Hussussian et al., Nat. Genet., 8(1):15-21, 1994.
Hutvagner et al., PLoS Biol., 2(4):E98, 2004.
Innis et al., Proc. Natl. Acad. Sci. USA, 85(24):9436-9440, 1988.
Inouye and Inouye, Nucleic Acids Res., 13:3101-3109, 1985.
Izant and Weintraub, Science, 229:345, 1985.
Ju et al., Gene Ther., 7(19):1672-1679, 2000.
Kahl et al., J. Mol. Biol., 299:75-89, 2000.
Kaihatsu et al., Biochem., 42(47):13996-4003, 2003.
Kaihatsu et al., Biochemistry, 43(45):14340-7, 2004.
Kaihatsu et al., Chem. Biol., 11:749-758, 2004.
Kaihatsu et al., Chem. Biol., 11:749-758, 2004.
Kamb et al., Nat. Genet., 8(1):23-26, 1994.
Kamb et al., Science, 2674:436-440, 1994.
Kashani-Sabet et al., Antisense Res. Dev., 2:3, 1992.
Kaur et al., Biochemistry, 45(23):7347-7355, 2006.
Kerr et al., Br. J. Cancer, 26(4):239-257, 1972.
Kiriazis and Kranias, Annu. Rev. Physiol., 62:321-351, 2000.
Koshkin et al., Bioorg. Med. Chem. Lett., 8(16):2219-22, 1998.
Krenz and Robbins, J. Am. Coll. Cardiol., 44:2390-2397, 2004.
Krützfeldt et al., 2005
Krützfeldt et al., Nature, 438:685-689, 2005.
Kumar et al., Biochem. Pharmacol., 55:775-783, 1998.
Kwoh et al., Proc. Natl. Acad. Sci. USA, 86:1173, 1989.
Lagos-Quintana et al., Science, 294(5543):853-858, 2001.
Larsen and Nielsen, Nucl. Acids Res., 24:458-463, 1996.
Lau et al., Science, 294(5543):858-862, 2001.
Lee and Ambros, Science, 294(5543):862-864, 2001.
Lee et al., ACS Symp. Ser., 752:184, 2000.
Levenson et al., Hum. Gene Ther., 9(8):1233-1236, 1998.
Li et al., Proc. Natl. Acad. Sci. USA, 102:19231-19236, 2005.
Lowes et al., J. Clin. Invest., 100(9):2315-2324, 1997.
MacBeath and Schreiber, Science, 289(5485):1760-1763, 2000.
Maurer et al., Mol. Membr. Biol., 16:129, 1999.
McGarry and Lindquist, Proc. Natl. Acad. Sci. USA, 83:399, 1986.
McMahon et al., Life Sci., 71(3):325-3, 2002.
Meister et al., Mol. Cell., 15:185-197, 2004.
Mitchell et al., J. Clin. Oncol., 8(5):856-869, 1990.
Miyata et al., Circ. Res., 86(4):386-390, 2000.
Mollegaard et al., Proc. Natl. Acad. Sci. USA, 91:3892-3895, 1994.
Mori et al., Cancer Res., 54(13):3396-3397, 1994.
Morita et al., Bioorg. Med. Chem., 11(10):2211-26, 2003.
Morris et al., Science, 305:1289-1292, 2004.
Morton et al., Arch. Surg., 127:392-399, 1992.
Nakamura et al., In: Handbook of Experimental Immunology (4^{th} Ed.), Weir et al., (eds). 1:27, Blackwell Scientific Publ., Oxford, 1987.
Nicolas and Rubenstein, In: Vectors: A survey of molecular cloning vectors and their uses, Rodriguez and Denhardt (Eds.), Stoneham: Butterworth, 494-513, 1988.
Nielsen et al., Science, 254:1497-1500, 1991.
Nobori et al., Nature (London), 368:753-756, 1995.
Ohara et al., Proc. Natl. Acad. Sci. USA, 86:5673-5677, 1989.
Ohkawa et al., Nucleic Acids Symp. Ser., 27:156, 1992.
Ojwang et al., Proc. Natl. Acad. Sci. USA, 89:10802, 1992.
Okamoto et al., Proc. Natl. Acad. Sci. USA, 91(23):11045-11049, 1994.
Orlow et al., Cancer Res, 54(11):2848-2851, 1994.
Orlow et al., Int. J. Oncol., 15(1):17-24, 1994.
Pandey and Mann, Nature, 405(6788):837-846, 2000.
Paroo and Corey, Trends Biotechnol., 22(8):390-4, 2004.
PCT Appln. PCT/US87/00880
PCT Appln. PCT/US89/01025
PCT Appln. WO 00/53722
PCT Appln. WO 01/38580
PCT Appln. WO 01/68255
PCT Appln. WO 03/020898
PCT Appln. WO 03/022421
PCT Appln. WO 03/023058
PCT Appln. WO 03/029485
PCT Appln. WO 03/040410
PCT Appln. WO 03/053586
PCT Appln. WO 03/066906
PCT Appln. WO 03/067217
PCT Appln. WO 03/076928
PCT Appln. WO 03/087297
PCT Appln. WO 03/091426
PCT Appln. WO 03/093810
PCT Appln. WO 03/100012
PCT Appln. WO 03/100448A1
PCT Appln. WO 04/020085
PCT Appln. WO 04/027093
PCT Appln. WO 05/021570
PCT Appln. WO 05/115481
PCT Appln. WO 05/121371
PCT Appln. WO 09/923,256
PCT Appln. WO 09/936,760
PCT Appln. WO 88/10315
PCT Appln. WO 89/06700
PCT Appln. WO 90/07641
PCT Appln. WO 93/17126
PCT Appln. WO 93/23569
PCT Appln. WO 94/02595
PCT Appln. WO 94/02595
PCT Appln. WO 94/14226
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 97/43450
PCT Appln. WO 98/39352
PCT Appln. WO 99/14226
PCT Appln. WO 99/14226
PCT Appln. WO 99/35505
Pietras et al., Oncogene, 17(17):2235-2249, 1998.
Prakash et al., J. Med. Chem., 48:4247-4253, 2005.
Qin et al., Proc. Natl. Acad. Sci. USA, 95(24):14411-14416, 1998.
Rand et al., Cell, 123:621-629, 2005.
Ravindranath and Morton, Intern. Rev. Immunol., 7: 303-329, 1991.
Ridgeway, In: Vectors: A Survey of Molecular Cloning Vectors and Their Uses, Rodriguez et al. (Eds.), Stoneham: Butterworth, 467-492, 1988.
Rosenberg et al., Ann. Surg. 210(4):474-548, 1989.
Rosenberg et al., N. Engl. J. Med., 319:1676, 1988.
Sambrook et al., In: Molecular cloning, Cold Spring Harbor Laboratory Press, Cold Spring Harbor, N.Y., 2001.
Sambrook et al., Molecular cloning. A laboratory manual; 2^{nd} Ed., Cold Spring Harbor Laboratory Press, 1989.
Sarver et al., Science, 247:1222-1225, 1990.
Scanlon et al., Proc. Natl. Acad. Sci. USA, 88:10591-10595, 1991.
Serrano et al., Nature, 366:704-707, 1993.
Serrano et al., Science, 267(5195):249-252, 1995.
Singh et al., Chem. Commun., 4:455-456, 1998
Singh et al., J. Org. Chem., 63:10035-10039, 1998.
Soutschek et al., Nature, 432(7014):173-178, 2004.
Summerton and Weller, Antisense Nucleic Acid Drug Dev.; 7(3):187-95, 1997.
Taira et al., Nucleic Acids Res., 19:5125, 1991.
Temin, In: Gene Transfer, Kucherlapati (Ed.), NY, Plenum Press, 149-188, 1986.
Thompson et al., Nature Genet., 9:444-450, 1995.
Tyler et al. Proc. Natl. Acad. Sci. USA, 8; 96(12):7053-8, 1999.
Tyler et al., Am. J. Physiol., 277(6 Pt 1):L1199-204, 1999.
United Kingdom Appln. 8 803 000
Urban-Klein, Gene Ther., 12:461-6, 2005.
Ventura et al., Nucleic Acids Res., 21:3249, 1993.
Wahlestedt et al., Proc. Natl. Acad. Sci. USA, 97(10):5633-5638, 2000.
Walker et al., Proc. Natl. Acad. Sci. USA, 89:392-396, 1992.
Weerasinghe et al., J. Virol., 65:5531, 1991.
Xu et al., Curr. Biol., 13(9):790-795, 2003.
Xu et al., DNA Cell Biol., 18, 85-95, 1999.
Zeng et al., Cancer Res., 62(13):3630-3635, 2002.
Zhang et al., J. Biol. Chem., 275:24436-24443, 2000.

1. A method of treating cancer in a subject comprising administering to said subject an effective amount of at least a first Six1-regulating miRNA.

2. The method of claim 1, wherein said miRNA targets the Six1 3′-UTR.

3. The method of claim 2, wherein the miRNA is selected from the group consisting of miR-185, miR-571 and miR-639.

4-6. (canceled)

7. The method of claim 3, further comprising administering at least two or all three of miR-185, miR-571 and miR-639.

8. (canceled)

9. The method of claim 1, wherein the miRNA is administered topically, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctivally, intravesicularly, mucosally, intrapericardially, intraumbilically, orally, by inhalation, by injection, by infusion, by continuous infusion, by localized perfusion bathing target cells directly, via a catheter, or via a lavage.

10. The method of claim 1, wherein said miRNA is administered more than once.

11. The method of claim 1, wherein said miRNA is administered by provision to said subject of an expression construct.

12-14. (canceled)

15. The method of claim 1, further comprising administering to said patient a second cancer therapy.

16. (canceled)

17. The method of claim 1, wherein the cancer is breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

18. The method of claim 1, wherein said miRNA comprises one or more stabilized non-natural nucleotides.

19. The method of claim 1, further comprising administering to said subject an antagomir of an miRNA that upregulates Six1 expression.

20. (canceled)

21. A method of identifying a cancer or a Six1-involved cancer in a subject comprising:
(a) obtaining a sample from said subject;
(b) assaying for the presence of one or more miRNAs in said sample selected from the group consisting of miR-106b, miR-93, miR-25, miR-375 and miR-622;
wherein an increase in said sample of one or more of miR-106b, miR-93, or miR-25, or a decrease in one or both of miR-375 or miR-622, as compared to a non-cancerous sample, indicates that said subject has a Six1-involved cancer.

22-27. (canceled)

28. The method of claim 21, further comprising administering to said subject an effective amount of at least a first Six1 miRNA when at least one of miR-106b, miR-93 and miR-25 are increased.

29. The method of claim 21, further comprising administering to said subject an effective amount of at least a first Six1 miRNA when at least one of miR-375 and miR-622 are decreased.

30-40. (canceled)

41. A method of treating cancer in a subject comprising administering to said subject an effective amount of an antagomir selected from miR-106b, miR-93, or miR-25.

42. The method of claim 41, further comprising administering at least two of or all three antagomirs of miR-106b, miR-93, and/or miR-25.

43. The method of claim 41, wherein the antagomir is administered topically, intravenously, intradermally, intraarterially, intraperitoneally, intralesionally, intracranially, intraarticularly, intraprostaticaly, intrapleurally, intratracheally, intraocularly, intranasally, intravitreally, intravaginally, intrarectally, intramuscularly, subcutaneously, subconjunctivally, intravesicularly, mucosally, intrapericardially, intraumbilically, orally, by inhalation, by injection, by infusion, by continuous infusion, by localized perfusion bathing target cells directly, via a catheter, or via a lavage.

44. (canceled)

45. The method of claim 41, wherein said antagomir is administered by provision to said subject of an expression construct.

46. (canceled)

49. The method of claim 41, further comprising administering to said patient a second cancer therapy.

50. The method of claim 41, wherein the cancer is breast cancer, lung cancer, prostate cancer, ovarian cancer, brain cancer, liver cancer, cervical cancer, colorectal cancer, renal cancer, skin cancer, head and neck cancer, bone cancer, esophageal cancer, bladder cancer, uterine cancer, lymphatic cancer, stomach cancer, pancreatic cancer, testicular cancer, lymphoma, or leukemia.

51. The method of claim 41, further comprising administering miR-185, miR-571 or miR-639 to said subject.

52-56. (canceled)
